SEQUENCE LISTING (155 sequences)

SEQ ID NO: 1 - protein, 15 aa, Artificial
  Synthetic peptide
REVQSKIGYGRQIIS

SEQ ID NO: 2 - protein, 24 aa, Artificial
  Synthetic peptide
ELMTGELPYSHINNRDQIIFMVGR

SEQ ID NO: 3 - protein, 21 aa, Artificial
  Synthetic peptide
TGSLPYSHIGSRDQIIFMVGR

SEQ ID NO: 4 - protein, 18 aa, Artificial
  Synthetic peptide
GIVLYELMTGELPYSHIN

SEQ ID NO: 5 - protein, 9 aa, Homo sapiens
  MOD_RES (7)..(7): Hyp
KGDTGPXGP

SEQ ID NO: 6 - protein, 9 aa, Homo sapiens
  MOD_RES (2)..(2): Hyp
SXGEAGRXG

SEQ ID NO: 7 - protein, 9 aa, Homo sapiens
VLNLGPITR

SEQ ID NO: 8 - protein, 11 aa, Homo sapiens
  MOD_RES (2)..(2): Hyp
AXGDKGESGPS

SEQ ID NO: 9 - protein, 12 aa, Homo sapiens
  MOD_RES (2)..(2): Hyp
PXGSAGAPGKDG

SEQ ID NO: 10 - protein, 11 aa, Homo sapiens
  MOD_RES (2)..(2): Hyp
SXGPDGKTGPX

SEQ ID NO: 11 - protein, 11 aa, Homo sapiens
  MOD_RES (7)..(7): Hyp
MGPRGPXGPXG

SEQ ID NO: 12 - protein, 12 aa, Homo sapiens
  MOD_RES (2)..(2): Hyp
GXGSDGKXGPXG

SEQ ID NO: 13 - protein, 11 aa, Homo sapiens
  MOD_RES (2)..(2): Hyp
AXGDRGEXGPX

SEQ ID NO: 14 - protein, 13 aa, Homo sapiens
  MOD_RES (3)..(3): Hyp
GGXGSDGKXGPXG

SEQ ID NO: 15 - protein, 13 aa, Homo sapiens
  MOD_RES (3)..(3): Hyp
GPXGXPGPPGPPS

SEQ ID NO: 16 - protein, 12 aa, Homo sapiens
  MOD_RES (2)..(2): Hyp
PXGEAGKXGEQG

SEQ ID NO: 17 - protein, 13 aa, Homo sapiens
  MOD_RES (5)..(5): Hyp
GPPGXXGXPGPPS

SEQ ID NO: 18 - protein, 10 aa, Homo sapiens
MIEQNTKSPL

SEQ ID NO: 19 - protein, 12 aa, Homo sapiens
  MOD_RES (8)..(8): Hyp
DDGEAGKXGRXG

SEQ ID NO: 20 - protein, 13 aa, Homo sapiens
  MOD_RES (3)..(3): Hyp
GPXGEAGKXGEQG

SEQ ID NO: 21 - protein, 13 aa, Homo sapiens
  MOD_RES (2)..(2): Hyp
AXGDRGEXGPPGP

SEQ ID NO: 22 - protein, 12 aa, Homo sapiens
DKGETGEQGDRG

SEQ ID NO: 23 - protein, 14 aa, Homo sapiens
  MOD_RES (2)..(2): Hyp
SXGPDGKTGPXGPA

SEQ ID NO: 24 - protein, 14 aa, Homo sapiens
  MOD_RES (2)..(2): Hyp
SXGSXGPDGKTGPX

SEQ ID NO: 25 - protein, 15 aa, Homo sapiens
  MOD_RES (13)..(13): Hyp
TGPGGDKGDTGPXGP

SEQ ID NO: 26 - protein, 15 aa, Homo sapiens
  MOD_RES (2)..(2): Hyp
SXGGXGSDGKXGPXG

SEQ ID NO: 27 - protein, 14 aa, Homo sapiens
DSGSSEEQGGSSRA

SEQ ID NO: 28 - protein, 14 aa, Homo sapiens
  MOD_RES (2)..(2): Hyp
AXGEDGRXGPXGPQ

SEQ ID NO: 29 - protein, 16 aa, Homo sapiens
  MOD_RES (2)..(2): Hyp
GXXGGXGSDGKXGPXG

SEQ ID NO: 30 - protein, 14 aa, Homo sapiens
  MOD_RES (4)..(4): Hyp
DGPXGRDGQXGHKG

SEQ ID NO: 31 - protein, 14 aa, Homo sapiens
DEAGSEADHEGTHS

SEQ ID NO: 32 - protein, 16 aa, Homo sapiens
  MOD_RES (2)..(2): Hyp
AXGKNGERGGXGGXGP

SEQ ID NO: 33 - protein, 13 aa, Homo sapiens
YTKKVPQVSTPTL

SEQ ID NO: 34 - protein, 13 aa, Homo sapiens
DQSRVLNLGPITR

SEQ ID NO: 35 - protein, 16 aa, Homo sapiens
  MOD_RES (10)..(10): Hyp
DGQPGAKGEXGDAGAK

SEQ ID NO: 36 - protein, 16 aa, Homo sapiens
  MOD_RES (4)..(4): Hyp
DGQXGAKGEXGDAGAK

SEQ ID NO: 37 - protein, 17 aa, Homo sapiens
  MOD_RES (4)..(4): Hyp
VGPXGXPGPPGPPGPPS

SEQ ID NO: 38 - protein, 17 aa, Homo sapiens
  MOD_RES (3)..(3): Hyp
GSXGSXGPDGKTGPPGX

SEQ ID NO: 39 - protein, 14 aa, Homo sapiens
GDSDDDEPPPLPRL

SEQ ID NO: 40 - protein, 14 aa, Homo sapiens
VIDQSRVLNLGPIT

SEQ ID NO: 41 - protein, 17 aa, Homo sapiens
  MOD_RES (4)..(4): Hyp
DGQXGAKGEXGDAGAKG

SEQ ID NO: 42 - protein, 15 aa, Homo sapiens
GSEADHEGTHSTKRG

SEQ ID NO: 43 - protein, 13 aa, Homo sapiens
YKRKANDESNEHS

SEQ ID NO: 44 - protein, 17 aa, Homo sapiens
  MOD_RES (5)..(5): Hyp
NDGAXGKNGERGGXGGX

SEQ ID NO: 45 - protein, 17 aa, Homo sapiens
TGLSMDGGGSPKGDVDP

SEQ ID NO: 46 - protein, 18 aa, Homo sapiens
  MOD_RES (2)..(2): Hyp
AXGGKGDAGAXGERGPXG

SEQ ID NO: 47 - protein, 17 aa, Homo sapiens
  MOD_RES (3)..(3): Hyp
GPXGEAGKXGEQGVXGD

SEQ ID NO: 48 - protein, 15 aa, Homo sapiens
SGDSDDDEPPPLPRL

SEQ ID NO: 49 - protein, 18 aa, Homo sapiens
  MOD_RES (4)..(4): Hyp
DGAXGKNGERGGXGGXGP

SEQ ID NO: 50 - protein, 18 aa, Homo sapiens
  MOD_RES (2)..(2): Hyp
MXGSXGGXGSDGKXGXPG

SEQ ID NO: 51 - protein, 18 aa, Homo sapiens
  MOD_RES (2)..(2): Hyp
SXGNIGPAGKEGPVGLXG

SEQ ID NO: 52 - protein, 19 aa, Homo sapiens
  MOD_RES (3)..(3): Hyp
GSXGSXGPDGKTGPXGPAG

SEQ ID NO: 53 - protein, 16 aa, Homo sapiens
AGSEADHEGTHSTKRG

SEQ ID NO: 54 - protein, 18 aa, Homo sapiens
  MOD_RES (3)..(3): Hyp
GLXGTGGPXGENGKPGEX

SEQ ID NO: 55 - protein, 18 aa, Homo sapiens
  MOD_RES (2)..(2): Hyp
KXGEQGVXGDLGAXGPSG

SEQ ID NO: 56 - protein, 16 aa, Homo sapiens
DEAGSEADHEGTHSTK

SEQ ID NO: 57 - protein, 18 aa, Homo sapiens
  MOD_RES (3)..(3): Hyp
GLXGTGGPXGENGKXGEX

SEQ ID NO: 58 - protein, 18 aa, Homo sapiens
  MOD_RES (2)..(2): Hyp
PXGEAGKXGEQGVPGDLG

SEQ ID NO: 59 - protein, 19 aa, Homo sapiens
  MOD_RES (3)..(3): Hyp
GPXGPXGKNGDDGEAGKXG

SEQ ID NO: 60 - protein, 19 aa, Homo sapiens
  MOD_RES (3)..(3): Hyp
GPXGEAGKXGEQGVXGDLG

SEQ ID NO: 61 - protein, 18 aa, Homo sapiens
EEAPSLRPAPPPISGGGY

SEQ ID NO: 62 - protein, 20 aa, Homo sapiens
GNDGAPGKNGERGGPGGPGP

SEQ ID NO: 63 - protein, 20 aa, Homo sapiens
  MOD_RES (15)..(15): Hyp
GEKGPSGEAGTAGPXGTXGP

SEQ ID NO: 64 - protein, 20 aa, Homo sapiens
  MOD_RES (3)..(3): Hyp
GLXGTGGPXGENGKPGEPGX

SEQ ID NO: 65 - protein, 20 aa, Homo sapiens
  MOD_RES (2)..(2): Hyp
GXPGEGRAGEXGTAGXTGXP

SEQ ID NO: 66 - protein, 17 aa, Homo sapiens
GSVIDQSRVLNLGPITR

SEQ ID NO: 67 - protein, 17 aa, Homo sapiens
DEAGSEADHEGTHSTKR

SEQ ID NO: 68 - protein, 20 aa, Homo sapiens
  MOD_RES (5)..(5): Hyp
NSGEXGAXGSKGDTGAKGEX

SEQ ID NO: 69 - protein, 19 aa, Homo sapiens
  MOD_RES (4)..(4): Hyp
EGSXGRDGSXGAKGDRGET

SEQ ID NO: 70 - protein, 21 aa, Homo sapiens
  MOD_RES (11)..(11): Hyp
DAGPAGPKGEXGSXGENGAXG

SEQ ID NO: 71 - protein, 19 aa, Homo sapiens
  MOD_RES (16)..(16): Hyp
DDGEAGKPGRPGERGXXGP

SEQ ID NO: 72 - protein, 20 aa, Homo sapiens
  MOD_RES (12)..(12): Hyp
GDDGEAGKPGRXGERGPXGP

SEQ ID NO: 73 - protein, 21 aa, Homo sapiens
  MOD_RES (1)..(1): modified Lysine
XGNDGAXGKNGERGGXGGXGP

SEQ ID NO: 74 - protein, 17 aa, Homo sapiens
EAIPMSIPPEVKFNKPF

SEQ ID NO: 75 - protein, 20 aa, Homo sapiens
  MOD_RES (8)..(8): modified Lysine
GDDGEAGXPGRXGERGPXGP

SEQ ID NO: 76 - protein, 20 aa, Homo sapiens
DGESGRPGRPGERGLPGPPG

SEQ ID NO: 77 - protein, 23 aa, Homo sapiens
  MOD_RES (3)..(3): Hyp
AGXPGPPGXXGTSGHXGSXGSXG

SEQ ID NO: 78 - protein, 22 aa, Homo sapiens
  MOD_RES (6)..(6): Hyl
GEPGGXGERGAXGEKGEGGXPG

SEQ ID NO: 79 - protein, 22 aa, Homo sapiens
  MOD_RES (2)..(2): Hyp
PXGEAGKXGEQGVXGDLGAPGP

SEQ ID NO: 80 - protein, 19 aa, Homo sapiens
SGSVIDQSRVLNLGPITRK

SEQ ID NO: 81 - protein, 21 aa, Homo sapiens
  MOD_RES (10)..(10): Hyp
NGDDGEAGKXGRXGERGPXGP

SEQ ID NO: 82 - protein, 23 aa, Homo sapiens
  MOD_RES (2)..(2): Hyp
GXXGEAGKPGEQGVPGDLGAPGX

SEQ ID NO: 83 - protein, 24 aa, Homo sapiens
  MOD_RES (5)..(5): Hyp
SNGNXGXPGPSGSPGKDGPXGXAG

SEQ ID NO: 84 - protein, 23 aa, Homo sapiens
  MOD_RES (6)..(6): Hyp
DGKTGXPGPAGQDGRPGPXGXXG

SEQ ID NO: 85 - protein, 23 aa, Homo sapiens
  MOD_RES (4)..(4): Hyp
NGEXGGKGERGAXGEKGEGGXPG

SEQ ID NO: 86 - protein, 23 aa, Homo sapiens
  MOD_RES (5)..(5): Hyp
AEGSXGRDGSXGAKGDRGETGPA

SEQ ID NO: 87 - protein, 25 aa, Homo sapiens
  MOD_RES (23)..(23): Hyp
ADGQPGAKGEPGDAGAKGDAGPXGP

SEQ ID NO: 88 - protein, 22 aa, Homo sapiens
  MOD_RES (9)..(9): Hyl
NGDDGEAGXPGRXGERGPXGPQ

SEQ ID NO: 89 - protein, 21 aa, Homo sapiens
  MOD_RES (5)..(5): Hyp
NDGPXGRDGQXGHKGERGYXG

SEQ ID NO: 90 - protein, 25 aa, Homo sapiens
  MOD_RES (6)..(6): Hyp
GNSGEXGAXGSKGDTGAKGEXGPVG

SEQ ID NO: 91 - protein, 23 aa, Homo sapiens
  MOD_RES (15)..(15): Hyp
GKNGDDGEAGKPGRXGERGPXGP

SEQ ID NO: 92 - protein, 25 aa, Homo sapiens
  MOD_RES (15)..(15): Hyp
GRTGDAGPVGPPGPXGXXGXPGPPS

SEQ ID NO: 93 - protein, 23 aa, Homo sapiens
  MOD_RES (12)..(12): Hyp
GKNGDDGEAGKXGRXGERGXPGP

SEQ ID NO: 94 - protein, 20 aa, Homo sapiens
IEQNTKSPLFMGKVVNPTQK

SEQ ID NO: 95 - protein, 26 aa, Homo sapiens
  MOD_RES (7)..(7): Hyp
KGDAGAXGAXXGKGDAGAXGERGXPG

SEQ ID NO: 96 - protein, 24 aa, Homo sapiens
  MOD_RES (5)..(5): Hyp
QNGEXGGKGERGAPGEKGEGGXXG

SEQ ID NO: 97 - protein, 26 aa, Homo sapiens
  MOD_RES (11)..(11): Hyp
ADGQPGAKGEXGDAGAKGDAGPPGXA

SEQ ID NO: 98 - protein, 26 aa, Homo sapiens
  MOD_RES (5)..(5): Hyp
ADGQXGAKGEXGDAGAKGDAGPXGPA

SEQ ID NO: 99 - protein, 26 aa, Homo sapiens
  MOD_RES (5)..(5): Hyp
ADGQXGAKGEXGDAGAKGDAGXXGPA

SEQ ID NO: 100 - protein, 25 aa, Homo sapiens
  MOD_RES (6)..(6): Hyp
GQNGEXGGKGERGAXGEKGEGGPXG

SEQ ID NO: 101 - protein, 26 aa, Homo sapiens
  MOD_RES (7)..(7): Hyp
KGNSGEXGAXGSKGDTGAKGEXGPVG

SEQ ID NO: 102 - protein, 24 aa, Homo sapiens
  MOD_RES (15)..(15): Hyp
GKNGDDGEAGKPGRXGERGPXGPQ

SEQ ID NO: 103 - protein, 24 aa, Homo sapiens
  MOD_RES (11)..(11): Hyl
GKNGDDGEAGXPGRXGERGPXGPQ

SEQ ID NO: 104 - protein, 21 aa, Homo sapiens
MIEQNTKSPLFMGKVVNPTQK

SEQ ID NO: 105 - protein, 21 aa, Homo sapiens
  MOD_RES (1)..(1): Met-oxidized
XIEQNTKSPLFXGKVVNPTQK

SEQ ID NO: 106 - protein, 27 aa, Homo sapiens
LDGAKGDAGPAGPKGEPGSPGENGAPG

SEQ ID NO: 107 - protein, 21 aa, Homo sapiens [sequence omitted]

SEQ ID NO: 108 - protein, 28 aa, Homo sapiens
  MOD_RES (11)..(11): Hyp
ADGQPGAKGEXGDAGAKGDAGPXGPAGP

SEQ ID NO: 109 - protein, 28 aa, Homo sapiens
  MOD_RES (5)..(5): Hyp
ADGQXGAKGEXGDAGAKGDAGXPGPAGP

SEQ ID NO: 110 - protein, 29 aa, Homo sapiens
  MOD_RES (7)..(7): Hyp
TGPIGPXGPAGAXGDKGESGPSGPAGPTG

SEQ ID NO: 111 - protein, 22 aa, Homo sapiens
  MOD_RES (2)..(2): Met-oxidized
LXIEQNTKSPLFMGKVVNPTQK

SEQ ID NO: 112 - protein, 29 aa, Homo sapiens
  MOD_RES (9)..(9): Hyp
GPPGADGQXGAKGEXGDAGAKGDAGXPGP

SEQ ID NO: 113 - protein, 22 aa, Homo sapiens
DAHKSEVAHRFKDLGEENFKAL

SEQ ID NO: 114 - protein, 29 aa, Homo sapiens
  MOD_RES (3)..(3): Hyp
GPXGADGQXGAKGEXGDAGAKGDAGXPGP

SEQ ID NO: 115 - protein, 24 aa, Homo sapiens
DEAGSEADHEGTHSTKRGHAKSRP

SEQ ID NO: 116 - protein, 28 aa, Homo sapiens
  MOD_RES (3)..(3): Hyp
GAXGQNGEXGGKGERGAXGEKGEGGPXG

SEQ ID NO: 117 - protein, 24 aa, Homo sapiens
DDILASPPRLPEPQPYPGAPHHSS

SEQ ID NO: 118 - protein, 28 aa, Homo sapiens
PEAEAEAEAGAGGEAAAEEGAAGRKARG

SEQ ID NO: 119 - protein, 30 aa, Homo sapiens
  MOD_RES (4)..(4): Hyp
AGPXGAXGAXGAPGPVGPAGKSGDRGETGP

SEQ ID NO: 120 - protein, 30 aa, Homo sapiens
  MOD_RES (4)..(4): Hyp
AGPXGAXGAXGAXGPVGPAGKSGDRGETGP

SEQ ID NO: 121 - protein, 29 aa, Homo sapiens
  MOD_RES (3)..(3): Hyp
QGXPGPSGEEGKRGPNGEAGSAGPPGXXG

SEQ ID NO: 122 - protein, 28 aa, Homo sapiens
GEPLDARGHGRPGGSGASEEALSPRGAG

SEQ ID NO: 123 - protein, 28 aa, Homo sapiens
  MOD_RES (8)..(8): Hyp
ERGEAGIXGVXGAKGEDGKDGSXGEXGA

SEQ ID NO: 124 - protein, 28 aa, Homo sapiens
  MOD_RES (14)..(14): Hyp
NRGERGSEGSPGHXGQXGXXGXPGAPGP

SEQ ID NO: 125 - protein, 28 aa, Homo sapiens
  MOD_RES (11)..(11): Hyp
NRGERGSEGSXGHXGQXGXXGPPGAPGX

SEQ ID NO: 126 - protein, 30 aa, Homo sapiens
  MOD_RES (11)..(11): Hyp
KNGETGPQGPXGPTGPGGDKGDTGPXGPQG

SEQ ID NO: 127 - protein, 24 aa, Homo sapiens
DAHKSEVAHRFKDLGEENFKALVL

SEQ ID NO: 128 - protein, 30 aa, Homo sapiens
  MOD_RES (7)..(7): Hyp
ERGSPGXAGPKGSXGEAGRXGEAGLXGAKG

SEQ ID NO: 129 - protein, 30 aa, Homo sapiens
  MOD_RES (17)..(17): Hyp
KEGGKGPRGETGPAGRXGEVGXPGPXGPAG

SEQ ID NO: 130 - protein, 30 aa, Homo sapiens
  MOD_RES (8)..(8): Hyp
ERGEAGIXGVXGAKGEDGKDGSPGEXGANG

SEQ ID NO: 131 - protein, 31 aa, Homo sapiens
  MOD_RES (14)..(14): Hyp
ESGREGAPGAEGSXGRDGSXGAKGDRGETGP

SEQ ID NO: 132 - protein, 31 aa, Homo sapiens
  MOD_RES (8)..(8): Hyp
ESGREGAXGAEGSXGRDGSXGAKGDRGETGP

SEQ ID NO: 133 - protein, 32 aa, Homo sapiens
  MOD_RES (9)..(9): Hyp
GESGREGAXGAEGSXGRDGSXGAKGDRGETGP

SEQ ID NO: 134 - protein, 32 aa, Homo sapiens
  MOD_RES (8)..(8): Hyp
ESGREGAXGAEGSXGRDGSXGAKGDRGETGPA

SEQ ID NO: 135 - protein, 33 aa, Homo sapiens
DGVSGGEGKGGSDGGGSHRKEGEEADAPGVIPG

SEQ ID NO: 136 - protein, 36 aa, Homo sapiens
  MOD_RES (5)..(5): Hyp
ADGQXGAKGEXGDAGAKGDAGPPGXAGPAGPPGXIG

SEQ ID NO: 137 - protein, 37 aa, Homo sapiens
  MOD_RES (12)..(12): Hyp
GADGQPGAKGEXGDAGAKGDAGPXGPAGXAGPPGPIG

SEQ ID NO: 138 - protein, 34 aa, Homo sapiens
  MOD_RES (3)..(3): Hyp
GEXGRDGVXGGXGMRGXXGSXGGXGSDGKXGPXG

SEQ ID NO: 139 - protein, 34 aa, Homo sapiens
  MOD_RES (2)..(2): Hyp
PXGESGREGAPGAEGSXGRDGSXGAKGDRGETGP

SEQ ID NO: 140 - protein, 34 aa, Homo sapiens
  MOD_RES (2)..(2): Hyp
PXGESGREGAXGAEGSXGRDGSXGAKGDRGETGP

SEQ ID NO: 141 - protein, 36 aa, Homo sapiens
  MOD_RES (8)..(8): Hyp
ENGKPGEXGXKGDAGAXGAXGGKGDAGAXGERGXPG

SEQ ID NO: 142 - protein, 36 aa, Homo sapiens
  MOD_RES (11)..(11): Hyp
RTGEVGAVGPXGFAGEXGPSGEAGTAGPXGTXGPQG

SEQ ID NO: 143 - protein, 35 aa, Homo sapiens
  MOD_RES (3)..(3): Hyp
GPXGESGREGAXGAEGSXGRDGSXGAKGDRGETGP

SEQ ID NO: 144 - protein, 33 aa, Homo sapiens
EEKAVADTRDQADGSRASVDSGSSEEQGGSSRA

SEQ ID NO: 145 - protein, 39 aa, Homo sapiens
  MOD_RES (2)..(2): Hyp
PXGADGQPGAKGEXGDAGAKGDAGXPGPAGPAGPPGPIG

SEQ ID NO: 146 - protein, 30 aa, Homo sapiens
EDPQGDAAQKTDTSHHDQDHPTFNKITPNL

SEQ ID NO: 147 - protein, 35 aa, Homo sapiens
FAEEKAVADTRDQADGSRASVDSGSSEEQGGSSRA

SEQ ID NO: 148 - protein, 32 aa, Homo sapiens
EDPQGDAAQKTDTSHHDQDHPTFNKITPNLAE

SEQ ID NO: 149 - protein, 37 aa, Homo sapiens
GRPEAQPPPLSSEHKEPVAGDAVPGPKDGSAPEVRGA

SEQ ID NO: 150 - protein, 37 aa, Homo sapiens
EEKAVADTRDQADGSRASVDSGSSEEQGGSSRALVST

SEQ ID NO: 151 - protein, 43 aa, Homo sapiens
ANGAPGNDGAKGDAGAPGAPGSQGAPGLQGMPGERGAAGLPGP

SEQ ID NO: 152 - protein, 45 aa, Homo sapiens
  MOD_RES (11)..(11): 3Hyp
ERGEQGPAGSXGFQGLXGXAGXXGEAGKXGEQGVPGDLGAPGPSG

SEQ ID NO: 153 - protein, 42 aa, Homo sapiens
EEKAVADTRDQADGSRASVDSGSSEEQGGSSRALVSTLVPLG

SEQ ID NO: 154 - protein, 48 aa, Homo sapiens
  MOD_RES (14)..(14): Hyp
ARGNDGARGSDGQXGXPGPXGTAGFXGSXGAKGEVGXAGSPGSNGAXG

SEQ ID NO: 155 - protein, 48 aa, Homo sapiens
LQGLPGTGGPPGENGKPGEPGPKGDAGAPGAPGGKGDAGAPGERGPPG

(19) United States
(12) Patent Application Publication
(10) Pub. No.: US 2015/0024970 A1
(43) Pub. Date: Jan. 22, 2015

(54) KIDNEY CELL CARCINOMA

(71) Applicant: Mosaiques Diagnostics and Therapeutics AG, Hannover (DE)

(72) Inventor: Harald Mischak, Sehnde (DE)

(21) Appl. No.: 14/503,617

(22) Filed: Oct. 1, 2014

Related U.S. Application Data
(63) Continuation of application No. 13/119,374, filed on Apr. 5, 2011, now abandoned.

(30) Foreign Application Priority Data
Sep. 17, 2008 (EP) 08164519.4

(51) Int. Cl. G01N 33/574; G01N 33/493

(52) U.S. Cl. CPC G01N 33/57438; G01N 33/493
USPC 506/12; 506/39

(57) ABSTRACT

The process for the diagnosis of a renal cell carcinoma comprises the step of determining the presence or absence or amplitude of at least three polypeptide markers in a urine sample, wherein said polypeptide markers are selected from the markers as characterized in Table 1 by molecular masses and migration times.

### CROSS REFERENCE TO RELATED APPLICATIONS

This application is continuation of copending U.S. patent application Ser. No. 13/119,374, which is a national stage filing of PCT application number PCT/EP2009/062072 filed on Sep. 17, 2009, which claims priority to European patent application serial number 08164519.4 filed on Sep. 17, 2008 to Mischak, entitled “Kidney Cell Carcinoma,” each of which is incorporated herein by reference.

The present invention relates to a process and a device for the diagnosis of renal cell carcinomas.

Worldwide, about 210,000 new cases of kidney cancer are diagnosed per year, with about 100,000 deaths per year. 90% of all kidney tumors are renal cell carcinomas (RCC), which form in the renal parenchyma. To date, there have been no diagnostic markers for RCC. Due to the little specific or even absent symptoms, a diagnosis is often made only in the advanced state. About 30% of the patients already have metastases at the time of the first diagnosis. According to a German study, RCC is diagnosed only by chance in 61% of the patients.

Therefore, there is a need for an early diagnosis, especially because metastatic RCC poorly responds to the usual chemotherapy or radiation therapy. The patients have an average survival time of 10 to 13 months and a five-year survival rate of less than 10%.

Novel drugs based on tyrosine kinase inhibitors promise improvement in the field of therapy, but nevertheless an early diagnosis is an essential precondition of a promising therapy.

A typical phenomenon in urogenital diseases is hematuria. It occurs in various kidney and bladder diseases without providing an unequivocal diagnostic means for distinguishing between, for example, ANCA-associated vasculitis, IgA nephropathy, bladder cancer and renal cancer.

B. Perroud et al.: Molecular Cancer 2006, 5: 64, describe renal cancer markers derived from eight samples from only four patients, so that the information is not statistically significant.

Medline PMID 15312509, Wu, D.-L. et al. describe four potential biomarkers.

In Technology in Cancer Research and Treatment 7 (2008), 155-160, the same authors also describe these four markers. The following measuring accuracy is stated: 4020±5, 4637±8, 5070±8 and 5500±11. The figure 4020±5 covers at least 185 peptides in the urine, the other figures cover 280, 289 and 100 peptides, respectively, so that an assignment between the disease and the stated masses is not possible.

Therefore, the information does not allow for a clear identification, and the quality of the markers ist questionable.

T. Kind et al. in Analytical Biochemistry 363 (2007), 185-195, describe metabolites for the identification of renal cancer. These are not peptides.

B. Delahunt et al. 38 (2007) in Human Pathology, 1372-1377, describe the collagen expression in kidney carcinomas. What is described is only the expression in the tissue, not in the urine, and diagnostic applications are not described either.

JP 2002022746 relates to a tumor marker suitable for the diagnosis of different carcinomas, such as brain tumors, ovarian carcinomas, renal carcinomas etc. Thus, it is not a specific marker for RCC.

Thus, there is still a need for diagnostic methods for recognizing renal cell carcinomas and for distinguishing between renal cell carcinomas and other diseases.

Surprisingly, the object can be achieved by a process for the diagnosis of a renal cell carcinoma, comprising the step of determining the presence or absence or amplitude of at least three polypeptide markers in a urine sample, wherein said polypeptide markers are selected from the markers as characterized in Table 1 by molecular masses and migration times.

TABLE 1

| No. | Mass (Da) | CE_t (min) |
|---|---|---|
| 1 | 840.4071 | 23.16555 |
| 2 | 858.3934 | 23.2367 |
| 3 | 860.3624 | 26.1404 |
| 4 | 868.4094 | 23.31203 |
| 5 | 883.41 | 23.25627 |
| 6 | 884.3214 | 24.85187 |
| 7 | 902.4133 | 20.84575 |
| 8 | 911.2648 | 34.34517 |
| 9 | 911.4349 | 25.87627 |
| 10 | 912.5167 | 20.05824 |
| 11 | 935.4465 | 23.68105 |
| 12 | 944.5103 | 21.24885 |
| 13 | 981.5851 | 24.79552 |
| 14 | 984.4547 | 24.92263 |
| 15 | 988.5208 | 22.44416 |
| 16 | 988.528 | 35.86765 |
| 17 | 994.4344 | 25.07426 |
| 18 | 1009.449 | 27.26775 |
| 19 | 1016.445 | 25.78512 |
| 20 | 1025.46 | 25.59517 |
| 21 | 1032.449 | 25.89738 |
| 22 | 1040.475 | 25.05015 |
| 23 | 1050.477 | 26.92478 |
| 24 | 1058.476 | 24.89302 |
| 25 | 1069.469 | 26.33201 |
| 26 | 1071.494 | 21.43072 |
| 27 | 1075.487 | 20.61147 |
| 28 | 1080.482 | 27.76981 |
| 29 | 1080.5 | 25.69417 |
| 30 | 1096.483 | 26.07573 |
| 31 | 1099.491 | 28.2422 |
| 32 | 1100.502 | 37.03674 |
| 33 | 1110.389 | 33.6302 |
| 34 | 1126.507 | 25.5171 |
| 35 | 1128.394 | 33.59201 |
| 36 | 1128.488 | 25.64931 |
| 37 | 1129.46 | 27.91491 |
| 38 | 1130.337 | 35.39296 |
| 39 | 1140.516 | 25.38464 |
| 40 | 1141.515 | 24.50651 |
| 41 | 1141.535 | 37.33278 |
| 42 | 1142.556 | 21.89118 |
| 43 | 1143.52 | 36.96738 |
| 44 | 1154.512 | 25.64497 |
| 45 | 1157.537 | 37.44405 |
| 46 | 1159.603 | 26.06505 |
| 47 | 1160.359 | 35.60058 |
| 48 | 1162.544 | 20.11196 |
| 49 | 1173.529 | 37.49036 |
| 50 | 1179.523 | 27.11006 |
| 51 | 1180.517 | 35.69966 |
| 52 | 1182.548 | 28.273 |
| 53 | 1186.53 | 22.39375 |
| 54 | 1187.358 | 35.68919 |
| 55 | 1191.517 | 36.17672 |
| 56 | 1199.576 | 21.9516 |
| 57 | 1204.597 | 21.94117 |
| 58 | 1211.542 | 25.82128 |
| 59 | 1216.537 | 24.24347 |
| 60 | 1217.529 | 35.78057 |
| 61 | 1234.563 | 27.37202 |
| 62 | 1247.523 | 22.00076 |
| 63 | 1262.465 | 38.233 |
| 64 | 1263.543 | 22.72857 |
| 65 | 1265.589 | 27.08673 |
| 66 | 1270.548 | 29.38075 |
| 67 | 1281.585 | 27.09018 |
| 68 | 1283.554 | 27.26645 |
| 69 | 1286.543 | 29.40513 |
| 70 | 1295.356 | 34.16189 |
| 71 | 1297.582 | 27.36504 |
| 72 | 1299.583 | 22.38183 |
| 73 | 1324.592 | 28.70152 |
| 74 | 1326.55 | 29.20429 |
| 75 | 1337.62 | 38.19948 |
| 76 | 1339.602 | 27.48587 |
| 77 | 1351.635 | 38.75677 |
| 78 | 1352.556 | 29.76547 |
| 79 | 1352.779 | 24.60145 |
| 80 | 1353.588 | 21.48296 |
| 81 | 1353.656 | 25.63162 |
| 82 | 1357.578 | 30.02141 |
| 83 | 1358.38 | 36.46108 |
| 84 | 1367.643 | 38.88257 |
| 85 | 1378.613 | 28.822 |
| 86 | 1383.593 | 27.62604 |
| 87 | 1405.635 | 20.13912 |
| 88 | 1406.635 | 28.15559 |
| 89 | 1407.657 | 37.22917 |
| 90 | 1413.55 | 25.55998 |
| 91 | 1422.597 | 21.72293 |
| 92 | 1422.677 | 28.14366 |
| 93 | 1425.587 | 22.31776 |
| 94 | 1435.703 | 22.54087 |
| 95 | 1438.667 | 27.87549 |
| 96 | 1440.562 | 24.30044 |
| 97 | 1449.641 | 21.85606 |
| 98 | 1451.693 | 22.55358 |
| 99 | 1460.804 | 22.76516 |
| 100 | 1466.653 | 28.51877 |
| 101 | 1467.795 | 23.8873 |
| 102 | 1467.807 | 24.68522 |
| 103 | 1469.668 | 23.69358 |
| 104 | 1482.666 | 22.46624 |
| 105 | 1485.674 | 23.76807 |
| 106 | 1486.683 | 21.15232 |
| 107 | 1487.652 | 29.62173 |
| 108 | 1491.739 | 39.83392 |
| 109 | 1496.684 | 30.37452 |
| 110 | 1508.678 | 29.33272 |
| 111 | 1510.654 | 28.27854 |
| 112 | 1510.682 | 20.16625 |
| 113 | 1521.688 | 30.52821 |
| 114 | 1522.712 | 22.89534 |
| 115 | 1523.841 | 29.75377 |
| 116 | 1524.654 | 20.02625 |
| 117 | 1526.69 | 23.91861 |
| 118 | 1542.692 | 23.96014 |
| 119 | 1549.696 | 39.48611 |
| 120 | 1567.702 | 20.19208 |
| 121 | 1576.6 | 26.37432 |
| 122 | 1576.743 | 19.50785 |
| 123 | 1580.886 | 24.84996 |
| 124 | 1583.701 | 23.26777 |
| 125 | 1586.738 | 28.88186 |
| 126 | 1588.706 | 30.15033 |
| 127 | 1592.697 | 22.1803 |
| 128 | 1594.726 | 23.05294 |
| 129 | 1594.762 | 40.21545 |
| 130 | 1595.704 | 30.00662 |
| 131 | 1608.684 | 22.34901 |
| 132 | 1608.73 | 30.93209 |
| 133 | 1623.727 | 24.12394 |
| 134 | 1624.546 | 37.72626 |
| 135 | 1627.695 | 29.45318 |
| 136 | 1634.799 | 29.71747 |
| 137 | 1635.76 | 30.33571 |
| 138 | 1636.699 | 20.03245 |
| 139 | 1636.741 | 30.25104 |
| 140 | 1638.728 | 20.22988 |
| 141 | 1640.581 | 23.24178 |
| 142 | 1640.678 | 28.04219 |
| 143 | 1651.79 | 40.66166 |
| 144 | 1664.746 | 29.81371 |
| 145 | 1666.775 | 30.66426 |
| 146 | 1668.805 | 40.46556 |
| 147 | 1669.689 | 21.46473 |
| 148 | 1671.539 | 37.6477 |
| 149 | 1679.699 | 22.60456 |
| 150 | 1680.752 | 30.02747 |
| 151 | 1692.798 | 30.88753 |
| 152 | 1693.762 | 20.50581 |
| 153 | 1696.718 | 23.94985 |
| 154 | 1706.777 | 22.68944 |
| 155 | 1708.785 | 30.59054 |
| 156 | 1714.548 | 37.84911 |
| 157 | 1716.657 | 20.17727 |
| 158 | 1725.591 | 38.32196 |
| 159 | 1728.771 | 36.767 |
| 160 | 1731.746 | 22.72137 |
| 161 | 1732.771 | 28.17527 |
| 162 | 1741.737 | 30.19875 |
| 163 | 1750.784 | 23.8278 |
| 164 | 1754.895 | 31.25885 |
| 165 | 1764.68 | 19.90654 |
| 166 | 1764.79 | 29.82516 |
| 167 | 1764.809 | 26.55983 |
| 168 | 1765.809 | 31.00023 |
| 169 | 1767.781 | 19.81004 |
| 170 | 1769.713 | 28.13518 |
| 171 | 1770.592 | 38.25859 |
| 172 | 1782.842 | 25.91265 |
| 173 | 1786.581 | 38.29337 |
| 174 | 1793.884 | 32.3738 |
| 175 | 1794.796 | 23.91532 |
| 176 | 1796.772 | 30.92154 |
| 177 | 1798.716 | 36.94821 |
| 178 | 1806.827 | 23.06136 |
| 179 | 1807.809 | 20.64857 |
| 180 | 1813.715 | 31.69499 |
| 181 | 1817.694 | 20.23435 |
| 182 | 1818.827 | 30.95312 |
| 183 | 1819.796 | 23.36311 |
| 184 | 1821.815 | 30.17125 |
| 185 | 1822.735 | 30.87417 |
| 186 | 1822.829 | 26.99979 |
| 187 | 1823.988 | 24.39771 |
| 188 | 1825.786 | 20.13497 |
| 189 | 1825.796 | 31.93096 |
| 190 | 1828.848 | 21.20199 |
| 191 | 1835.709 | 19.91313 |
| 192 | 1837.8 | 30.55694 |
| 193 | 1838.818 | 27.0581 |
| 194 | 1840.836 | 41.17953 |
| 195 | 1841.751 | 35.65976 |
| 196 | 1843.779 | 30.63276 |
| 197 | 1848.806 | 30.80986 |
| 198 | 1858.839 | 24.2646 |
| 199 | 1859.828 | 24.41139 |
| 200 | 1860.461 | 34.21703 |
| 201 | 1860.826 | 21.40014 |
| 202 | 1865.808 | 32.98199 |
| 203 | 1874.831 | 30.82379 |
| 204 | 1876.866 | 22.20126 |
| 205 | 1876.842 | 23.38023 |
| 206 | 1878.59 | 30.77853 |
| 207 | 1878.792 | 20.7224 |
| 208 | 1880.895 | 43.9095 |
| 209 | 1885.651 | 38.81996 |
| 210 | 1889.868 | 33.0683 |
| 211 | 1892.869 | 22.19629 |
| 212 | 1892.973 | 24.55813 |
| 213 | 1915.909 | 31.29872 |
| 214 | 1916.768 | 20.32244 |
| 215 | 1916.946 | 34.19299 |
| 216 | 1925.815 | 23.1958 |
| 217 | 1933.877 | 21.62452 |
| 218 | 1934.786 | 19.94225 |
| 219 | 1938.881 | 21.38561 |
| 220 | 1942.837 | 30.96243 |
| 221 | 1943.007 | 24.9416 |
| 222 | 1945.004 | 33.70855 |
| 223 | 1949.889 | 21.65736 |
| 224 | 1955.882 | 28.11064 |
| 225 | 1959.006 | 25.01828 |
| 226 | 1963.88 | 31.74253 |
| 227 | 1969.838 | 25.22952 |
| 228 | 1976.882 | 32.38465 |
| 229 | 1977.918 | 32.19436 |
| 230 | 1991.941 | 22.04607 |
| 231 | 1996.786 | 20.9757 |
| 232 | 2003.939 | 24.61864 |
| 233 | 2007.945 | 22.10222 |
| 234 | 2008.902 | 32.2865 |
| 235 | 2013.893 | 31.75577 |
| 236 | 2016.044 | 21.3587 |
| 237 | 2023.908 | 21.48129 |
| 238 | 2029.853 | 20.39044 |
| 239 | 2030.93 | 32.60772 |
| 240 | 2034.995 | 40.18541 |
| 241 | 2039.129 | 21.77864 |
| 242 | 2041.979 | 28.49748 |
| 243 | 2042.071 | 25.1431 |
| 244 | 2048.927 | 24.46324 |
| 245 | 2055.939 | 25.43797 |
| 246 | 2058.937 | 23.15082 |
| 247 | 2063.932 | 21.94861 |
| 248 | 2064.918 | 24.45992 |
| 249 | 2067.818 | 20.62077 |
| 250 | 2076.945 | 21.77894 |
| 251 | 2087.966 | 32.91176 |
| 252 | 2088.875 | 23.6675 |
| 253 | 2089.958 | 39.51667 |
| 254 | 2109.923 | 24.06903 |
| 255 | 2117.934 | 32.96822 |
| 256 | 2128.983 | 26.96859 |
| 257 | 2132.909 | 25.82955 |
| 258 | 2135.958 | 25.80025 |
| 259 | 2137.942 | 21.79294 |
| 260 | 2152.979 | 32.81957 |
| 261 | 2156.974 | 22.21732 |
| 262 | 2168.967 | 32.9057 |
| 263 | 2184.568 | 35.08425 |
| 264 | 2185.983 | 25.87921 |
| 265 | 2187.949 | 39.78272 |
| 266 | 2188.999 | 26.88501 |
| 267 | 2191.99 | 22.39346 |
| 268 | 2194.971 | 20.16669 |
| 269 | 2198.999 | 22.31204 |
| 270 | 2208.891 | 31.70258 |
| 271 | 2211.975 | 33.23364 |
| 272 | 2216.03 | 33.83058 |
| 273 | 2226.992 | 26.27752 |
| 274 | 2233.045 | 20.51498 |
| 275 | 2235.045 | 34.16645 |
| 276 | 2236.982 | 27.13894 |
| 277 | 2249.042 | 20.532 |
| 278 | 2256.973 | 33.5541 |
| 279 | 2257.869 | 35.92739 |
| 280 | 2258.185 | 22.08701 |
| 281 | 2264.035 | 22.67238 |
| 282 | 2265.974 | 33.73205 |
| 283 | 2266.021 | 22.1634 |
| 284 | 2272.226 | 23.87656 |
| 285 | 2274.044 | 33.50617 |
| 286 | 2276.023 | 27.23118 |
| 287 | 2277.008 | 27.22479 |
| 288 | 2280.944 | 36.21864 |
| 289 | 2282.016 | 22.23937 |
| 290 | 2289.039 | 33.59018 |
| 291 | 2292.019 | 27.28272 |
| 292 | 2302.198 | 26.13343 |
| 293 | 2308.017 | 27.33705 |
| 294 | 2310.056 | 41.31365 |
| 295 | 2318.21 | 26.30227 |
| 296 | 2321.165 | 22.05962 |
| 297 | 2323.043 | 22.36064 |
| 298 | 2339 | 34.00721 |
| 299 | 2349.043 | 27.36547 |
| 300 | 2355.085 | 22.74837 |
| 301 | 2356.659 | 35.533 |
| 302 | 2361.108 | 20.79425 |
| 303 | 2371.084 | 22.7883 |
| 304 | 2377.101 | 20.79967 |
| 305 | 2385.054 | 33.94877 |
| 306 | 2389.241 | 22.39921 |
| 307 | 2394.08 | 23.63506 |
| 308 | 2405.222 | 22.47371 |
| 309 | 2407.092 | 27.67152 |
| 310 | 2414.154 | 19.57439 |
| 311 | 2414.626 | 35.61674 |
| 312 | 2420.998 | 34.86081 |
| 313 | 2421.228 | 22.5266 |
| 314 | 2423.092 | 27.66597 |
| 315 | 2423.324 | 21.07748 |
| 316 | 2427.184 | 19.58138 |
| 317 | 2430.098 | 28.32809 |
| 318 | 2446.092 | 28.37261 |
| 319 | 2471.155 | 34.77354 |
| 320 | 2485.125 | 34.4072 |
| 321 | 2490.231 | 24.68039 |
| 322 | 2501.119 | 34.38645 |
| 323 | 2507.126 | 22.80579 |
| 324 | 2518.312 | 22.7888 |
| 325 | 2525.195 | 27.73635 |
| 326 | 2529.135 | 28.24742 |
| 327 | 2534.298 | 22.86796 |
| 328 | 2540.264 | 19.67573 |
| 329 | 2544.128 | 28.25992 |
| 330 | 2545.12 | 28.20161 |
| 331 | 2547.986 | 21.4417 |
| 332 | 2548.286 | 35.15708 |
| 333 | 2559.18 | 19.40742 |
| 334 | 2563.147 | 21.21006 |
| 335 | 2564.15 | 22.97496 |
| 336 | 2565.143 | 23.74013 |
| 337 | 2570.19 | 42.56018 |
| 338 | 2574.009 | 32.80843 |
| 339 | 2576.124 | 34.25753 |
| 340 | 2577.246 | 24.66592 |
| 341 | 2580.141 | 22.9806 |
| 342 | 2582.171 | 23.66997 |
| 343 | 2583.149 | 23.68335 |
| 344 | 2583.199 | 28.31444 |
| 345 | 2584.234 | 35.18279 |
| 346 | 2587.195 | 21.09996 |
| 347 | 2589.056 | 22.56216 |
| 348 | 2596.233 | 34.89552 |
| 349 | 2599.19 | 28.27509 |
| 350 | 2636.2 | 24.39431 |
| 351 | 2639.289 | 21.41891 |
| 352 | 2642.214 | 27.69602 |
| 353 | 2644.217 | 21.15259 |
| 354 | 2654.193 | 23.92493 |
| 355 | 2663.204 | 23.50693 |
| 356 | 2679.197 | 23.52889 |
| 357 | 2682.143 | 22.49183 |
| 358 | 2686.336 | 29.34243 |
| 359 | 2687.219 | 28.98551 |
| 360 | 2695.198 | 23.52252 |
| 361 | 2702.213 | 38.0799 |
| 362 | 2710.323 | 35.07814 |
| 363 | 2713.234 | 29.22274 |
| 364 | 2726.283 | 42.93932 |
| 365 | 2733.781 | 34.1596 |
| 366 | 2742.251 | 42.14319 |
| 367 | 2742.25 | 28.98028 |
| 368 | 2748.788 | 36.38231 |
| 369 | 2752.413 | 19.8887 |
| 370 | 2754.272 | 29.67942 |
| 371 | 2756.268 | 35.24034 |
| 372 | 2761.315 | 21.49226 |
| 373 | 2767.323 | 21.6729 |
| 374 | 2770.246 | 29.3928 |
| 375 | 2802.82 | 36.33852 |
| 376 | 2808.343 | 24.39073 |
| 377 | 2809.235 | 24.40412 |
| 378 | 2818.803 | 36.27764 |
| 379 | 2841.256 | 24.53535 |
| 380 | 2887.347 | 35.71764 |
| 381 | 2903.361 | 35.70914 |
| 382 | 2907.352 | 35.95513 |
| 383 | 2914.379 | 24.33308 |
| 384 | 2923.432 | 36.91513 |
| 385 | 2926.3 | 22.21827 |
| 386 | 2939.149 | 33.76711 |
| 387 | 2942.299 | 22.23281 |
| 388 | 2973.452 | 24.3704 |
| 389 | 2977.373 | 29.11797 |
| 390 | 2987.348 | 38.54569 |
| 391 | 2989.451 | 24.42708 |
| 392 | 2999.285 | 22.24535 |
| 393 | 3002.238 | 23.80085 |
| 394 | 3007.407 | 20.95133 |
| 395 | 3013.292 | 22.2946 |
| 396 | 3021.351 | 23.41584 |
| 397 | 3023.407 | 20.94963 |
| 398 | 3031.427 | 36.03612 |
| 399 | 3041.375 | 29.98314 |
| 400 | 3058.378 | 24.82364 |
| 401 | 3064.322 | 20.57415 |
| 402 | 3081.416 | 29.83057 |
| 403 | 3091.436 | 28.39639 |
| 404 | 3092.464 | 31.24934 |
| 405 | 3108.454 | 31.28399 |
| 406 | 3137.411 | 30.34616 |
| 407 | 3139.487 | 29.48159 |
| 408 | 3145.459 | 38.89295 |
| 409 | 3149.46 | 31.24549 |
| 410 | 3152.34 | 24.55108 |
| 411 | 3157.128 | 34.69761 |
| 412 | 3165.462 | 31.32057 |
| 413 | 3166.271 | 22.05894 |
| 414 | 3168.357 | 24.70453 |
| 415 | 3193.382 | 22.64363 |
| 416 | 3205.273 | 19.65755 |
| 417 | 3209.407 | 22.67502 |
| 418 | 3256.527 | 33.03419 |
| 419 | 3258.463 | 22.91476 |
| 420 | 3264.556 | 25.75167 |
| 421 | 3264.528 | 30.65823 |
| 422 | 3266.417 | 22.74809 |
| 423 | 3280.556 | 25.81551 |
| 424 | 3281.434 | 36.0914 |
| 425 | 3295.533 | 25.45276 |
| 426 | 3303.501 | 30.88648 |
| 427 | 3314.431 | 20.14047 |
| 428 | 3318.546 | 30.99198 |
| 429 | 3333.719 | 23.83213 |
| 430 | 3338.463 | 23.58844 |
| 431 | 3343.569 | 31.85325 |
| 432 | 3349.42 | 35.89379 |
| 433 | 3356.518 | 22.03026 |
| 434 | 3359.578 | 31.89787 |
| 435 | 3361.556 | 24.24354 |
| 436 | 3375.574 | 31.91691 |
| 437 | 3385.547 | 25.48872 |
| 438 | 3401.596 | 25.47106 |
| 439 | 3401.66 | 23.49281 |
| 440 | 3416.602 | 36.84899 |
| 441 | 3421.555 | 25.99412 |
| 442 | 3426.31 | 27.69928 |
| 443 | 3432.593 | 32.04683 |
| 444 | 3478.434 | 41.74144 |
| 445 | 3510.6 | 40.24415 |
| 446 | 3530.645 | 26.12897 |
| 447 | 3556.588 | 23.95474 |
| 448 | 3556.603 | 22.63602 |
| 449 | 3559.709 | 24.92127 |
| 450 | 3589.683 | 25.03383 |
| 451 | 3596.698 | 21.49245 |
| 452 | 3616.724 | 33.18799 |
| 453 | 3630.443 | 21.77706 |
| 454 | 3635.661 | 31.79329 |
| 455 | 3657.665 | 40.71091 |
| 456 | 3669.666 | 24.16841 |
| 457 | 3685.833 | 22.19635 |
| 458 | 3718.721 | 32.4816 |
| 459 | 3719.734 | 22.49869 |
| 460 | 3722.724 | 22.0386 |
| 461 | 3734.721 | 32.49717 |
| 462 | 3738.721 | 24.76482 |
| 463 | 3745.661 | 26.68822 |
| 464 | 3759.851 | 19.41137 |
| 465 | 3774.719 | 22.93709 |
| 466 | 3775.748 | 25.58515 |
| 467 | 3802.714 | 32.43992 |
| 468 | 3816.753 | 21.96327 |
| 469 | 3839.813 | 19.70303 |
| 470 | 3858.843 | 25.85393 |
| 471 | 3870.814 | 33.49116 |
| 472 | 3886.83 | 33.53763 |
| 473 | 3891.752 | 24.52856 |
| 474 | 3927.821 | 33.59714 |
| 475 | 3932.864 | 25.93393 |
| 476 | 3943.83 | 33.62845 |
| 477 | 3945.911 | 22.03043 |
| 478 | 3968.597 | 21.09379 |
| 479 | 3986.65 | 20.60164 |
| 480 | 3996.658 | 20.92089 |
| 481 | 4002.618 | 20.65664 |
| 482 | 4008.81 | 23.42187 |
| 483 | 4015.997 | 28.07419 |
| 484 | 4024.865 | 33.26167 |
| 485 | 4043.639 | 20.38493 |
| 486 | 4044.915 | 26.36705 |
| 487 | 4083.799 | 31.25574 |
| 488 | 4097.87 | 24.61058 |
| 489 | 4121.881 | 23.55676 |
| 490 | 4169.926 | 33.58317 |
| 491 | 4217.975 | 26.05299 |
| 492 | 4251.984 | 28.76518 |
| 493 | 4305.937 | 28.82961 |
| 494 | 4321.941 | 25.19911 |
| 495 | 4352.86 | 20.16808 |
| 496 | 4404.842 | 20.66586 |
| 497 | 4409.888 | 20.00095 |
| 498 | 4436.083 | 26.31772 |
| 499 | 4549.146 | 26.55078 |
| 500 | 4671.824 | 23.27783 |
| 501 | 4771.071 | 20.19867 |
| 502 | 4817.155 | 23.90309 |
| 503 | 4833.145 | 23.92431 |
| 504 | 4863.156 | 26.73903 |
| 505 | 4960.418 | 20.61115 |
| 506 | 5043.131 | 26.60105 |
| 507 | 5213.091 | 22.43408 |
| 508 | 5510.356 | 27.07841 |
| 509 | 5574.253 | 23.20092 |
| 510 | 6236.907 | 21.066 |
| 511 | 6541.758 | 20.67838 |
| 512 | 8289.341 | 19.47861 |
| 513 | 8837.408 | 21.0634 |
| 514 | 8853.766 | 21.09699 |
| 515 | 8917.251 | 22.54506 |
| 516 | 9866.536 | 20.86863 |
| 517 | 10341.97 | 22.98239 |

In particular, the process is suitable for differential diagnosis for distinguishing between a renal cell carcinoma and other diseases selected from chronic kidney diseases, such as ANCA-associated vasculitis, IgA nephropathy, renal calculi and bladder cancer.

The following reference values can be employed for evaluating the measured presence or absence or amplitudes of the markers:

| | | | Mean | | Mean | | | | Mean | |
|---|---|---|---|---|---|---|---|---|---|---|
| | | Freq. | (median) | Freq. | (median) | Freq. | Mean | Freq. | (median) | |
| Mass | CE_t | RCC | RCC | Control | control | Bca | (median) Bca | CKD | CKD | AUC |
| 840.4071 | 23.16555 | 0.18 | 1.54(1.60) | 0.06 | 1.41(1.54) | 0.51 | 1.95(1.96) | 0.18 | 1.60(1.65) | 0.707174 |
| 858.3934 | 23.2367 | 0.4 | 1.97(1.97) | 0.26 | 1.53(1.59) | 0.7 | 2.24(2.33) | 0.58 | 1.96(1.92) | <0.7 |
| 860.3624 | 26.1404 | 0.19 | 1.95(2.02) | 0.26 | 1.67(1.63) | 0.5 | 2.27(2.32) | 0.3 | 2.10(2.19) | 0.756667 |
| 868.4094 | 23.31203 | 0.41 | 1.77(1.88) | 0.16 | 1.36(1.36) | 0.65 | 2.00(2.07) | 0.52 | 2.01(2.01) | <0.7 |
| 883.41 | 23.25627 | 0.31 | 1.55(1.61) | 0.09 | 1.27(1.23) | 0.56 | 1.88(1.93) | 0.24 | 1.79(1.73) | <0.7 |
| 884.3214 | 24.85187 | 0.58 | 2.11(2.10) | 0.63 | 1.86(1.88) | 0.87 | 2.37(2.39) | 0.68 | 2.16(2.23) | 0.706345 |
| 902.4133 | 20.84575 | 0.73 | 2.09(2.10) | 0.8 | 2.15(2.14) | 0.94 | 2.46(2.50) | 0.84 | 2.54(2.64) | <0.7 |
| 911.2648 | 34.34517 | 0.63 | 2.49(2.64) | 0.66 | 2.41(2.52) | 0.92 | 2.86(2.90) | 0.8 | 2.67(2.69) | <0.7 |
| 911.4349 | 25.87627 | 0.52 | 1.97(1.91) | 0.5 | 1.94(2.00) | 0.77 | 2.35(2.37) | 0.54 | 2.09(2.20) | <0.7 |
| 912.5167 | 20.05824 | 0.25 | 2.03(2.04) | 0.04 | 1.85(1.77) | 0.3 | 1.97(1.86) | 0.5 | 3.12(3.21) | 0.713333 |
| 935.4465 | 23.68105 | 0.2 | 1.68(1.64) | 0.1 | 1.51(1.49) | 0.54 | 1.83(1.68) | 0.24 | 1.79(1.73) | <0.7 |
| 944.5103 | 21.24885 | 0.74 | 2.29(2.39) | 0.63 | 2.41(2.39) | 0.65 | 2.62(2.74) | 0.54 | 2.47(2.55) | <0.7 |
| 981.5851 | 24.79552 | 0.7 | 2.35(2.25) | 0.8 | 2.25(2.26) | 0.7 | 2.55(2.62) | 0.56 | 2.42(2.48) | 0.747939 |
| 984.4547 | 24.92263 | 0.38 | 1.61(1.67) | 0.52 | 1.84(1.87) | 0.63 | 1.88(1.89) | 0.26 | 1.86(1.81) | <0.7 |
| 988.5208 | 22.44416 | 0.7 | 2.14(2.16) | 0.53 | 2.20(2.22) | 0.5 | 2.30(2.37) | 0.4 | 2.37(2.39) | <0.7 |
| 988.528 | 35.86765 | 0.31 | 1.67(1.63) | 0.13 | 1.37(1.37) | 0.43 | 1.95(1.98) | 0.24 | 1.88(2.04) | <0.7 |
| 994.4344 | 25.07426 | 0.33 | 1.60(1.55) | 0.36 | 1.83(1.90) | 0.52 | 1.99(1.98) | 0.36 | 1.99(1.91) | <0.7 |
| 1009.449 | 27.26775 | 0.25 | 1.65(1.62) | 0.19 | 1.97(1.99) | 0.46 | 2.09(2.11) | 0.34 | 2.06(2.15) | 0.706737 |
| 1016.445 | 25.78512 | 0.96 | 2.46(2.52) | 0.97 | 2.71(2.77) | 0.94 | 2.94(3.00) | 0.96 | 2.95(2.99) | 0.763258 |
| 1025.46 | 25.59517 | 0.52 | 1.94(2.06) | 0.4 | 2.15(2.20) | 0.51 | 2.45(2.54) | 0.14 | 2.02(2.06) | <0.7 |
| 1032.449 | 25.89738 | 0.34 | 1.84(1.85) | 0.54 | 2.02(2.03) | 0.67 | 2.20(2.19) | 0.52 | 2.27(2.32) | 0.727717 |
| 1040.475 | 25.05015 | 0.6 | 1.98(2.03) | 0.46 | 2.02(2.05) | 0.63 | 2.40(2.44) | 0.38 | 2.32(2.42) | <0.7 |
| 1050.477 | 26.92478 | 0.71 | 1.97(2.03) | 0.75 | 2.31(2.28) | 0.81 | 2.42(2.43) | 0.66 | 2.16(2.16) | 0.719660 |
| 1058.476 | 24.89302 | 0.19 | 2.97(3.07) | 0.21 | 2.74(2.91) | 0.32 | 2.68(2.97) | 0.72 | 2.91(2.97) | 0.736077 |
| 1069.469 | 26.33201 | 0.44 | 1.83(1.78) | 0.35 | 1.94(1.97) | 0.48 | 2.26(2.32) | 0.66 | 2.25(2.24) | <0.7 |
| 1071.494 | 21.43072 | 0.29 | 1.73(1.77) | 0.49 | 1.96(2.01) | 0.41 | 2.04(2.05) | 0.26 | 1.83(1.89) | 0.710298 |
| 1075.487 | 20.61147 | 0.32 | 2.08(2.08) | 0.55 | 2.34(2.40) | 0.23 | 2.07(2.10) | 0.14 | 1.90(1.82) | <0.7 |
| 1080.482 | 27.76981 | 0.04 | 1.99(1.98) | 0.28 | 1.97(2.02) | 0.29 | 2.06(2.07) | 0.12 | 1.49(1.69) | 0.803870 |
| 1080.5 | 25.69417 | 0.35 | 1.87(1.91) | 0.17 | 1.95(2.05) | 0.38 | 2.27(2.25) | 0.16 | 2.22(2.24) | <0.7 |
| 1096.483 | 26.07573 | 0.86 | 3.32(3.40) | 0.87 | 3.43(3.50) | 0.82 | 3.62(3.67) | 0.66 | 3.15(3.32) | <0.7 |
| 1099.491 | 28.2422 | 0.81 | 2.12(2.19) | 0.93 | 2.41(2.47) | 0.8 | 2.53(2.56) | 0.74 | 2.34(2.38) | 0.807143 |
| 1100.502 | 37.03674 | 0.31 | 1.68(1.60) | 0.26 | 1.77(1.69) | 0.45 | 2.08(2.07) | 0.26 | 2.17(2.14) | 0.749097 |
| 1110.389 | 33.6302 | 0.51 | 2.41(2.43) | 0.81 | 2.68(2.72) | 0.45 | 2.66(2.74) | 0.54 | 2.58(2.66) | 0.741621 |
| 1126.507 | 25.5171 | 0.34 | 1.86(1.92) | 0.35 | 2.03(2.10) | 0.62 | 2.19(2.21) | 0.62 | 2.16(2.11) | <0.7 |
| 1128.394 | 33.59201 | 0.79 | 2.72(2.87) | 0.92 | 3.03(3.02) | 0.88 | 3.13(3.12) | 0.84 | 3.01(3.07) | <0.7 |
| 1128.488 | 25.64931 | 0.52 | 1.81(1.77) | 0.65 | 2.12(2.10) | 0.69 | 2.29(2.35) | 0.7 | 2.22(2.36) | 0.728860 |
| 1129.46 | 27.91491 | 0.57 | 1.96(2.04) | 0.69 | 2.16(2.25) | 0.7 | 2.28(2.27) | 0.54 | 2.07(2.24) | 0.718966 |
| 1130.337 | 35.39296 | 0.63 | 2.43(2.54) | 0.7 | 2.59(2.76) | 0.93 | 2.71(2.75) | 0.68 | 2.69(2.87) | 0.722345 |
| 1140.516 | 25.38464 | 0.27 | 1.87(1.78) | 0.28 | 2.19(2.18) | 0.45 | 2.11(2.11) | 0.46 | 2.13(2.08) | <0.7 |
| 1141.515 | 24.50651 | 0.35 | 1.85(1.85) | 0.58 | 2.16(2.15) | 0.55 | 2.26(2.31) | 0.56 | 2.07(2.06) | <0.7 |
| 1141.535 | 37.33278 | 0.5 | 1.98(2.04) | 0.4 | 1.99(2.01) | 0.48 | 2.30(2.34) | 0.46 | 2.07(2.19) | 0.722724 |
| 1142.556 | 21.89118 | 0.64 | 1.97(2.00) | 0.54 | 1.99(2.01) | 0.54 | 2.29(2.33) | 0.24 | 2.41(2.40) | <0.7 |
| 1143.52 | 36.96738 | 0.54 | 2.08(2.11) | 0.5 | 2.10(2.07) | 0.53 | 2.41(2.48) | 0.44 | 2.26(2.28) | 0.812068 |
| 1154.512 | 25.64497 | 0.5 | 2.17(2.20) | 0.65 | 2.37(2.39) | 0.7 | 2.39(2.45) | 0.74 | 2.44(2.54) | 0.720453 |
| 1157.537 | 37.44405 | 0.89 | 2.51(2.52) | 0.87 | 2.54(2.50) | 0.85 | 2.94(2.95) | 0.66 | 2.81(2.85) | 0.825373 |
| 1159.603 | 26.06505 | 0.5 | 2.43(2.41) | 0.76 | 2.64(2.64) | 0.54 | 2.23(2.18) | 0.48 | 2.68(2.79) | <0.7 |
| 1160.359 | 35.60058 | 0.93 | 2.82(2.73) | 0.98 | 2.96(3.03) | 0.97 | 3.21(3.18) | 0.86 | 3.16(3.22) | 0.724241 |
| 1162.544 | 20.11196 | 0.46 | 1.97(1.97) | 0.61 | 2.20(2.19) | 0.62 | 2.20(2.20) | 0.28 | 2.17(2.16) | <0.7 |
| 1173.529 | 37.49036 | 0.39 | 1.89(1.80) | 0.44 | 2.02(2.06) | 0.47 | 2.19(2.15) | 0.28 | 2.20(2.30) | <0.7 |
| 1179.523 | 27.11006 | 0.82 | 2.50(2.56) | 0.96 | 2.72(2.71) | 0.75 | 2.67(2.71) | 0.92 | 2.75(2.81) | 0.707825 |
| 1180.517 | 35.69966 | 0.35 | 2.46(2.43) | 0.56 | 2.51(2.59) | 0.53 | 2.64(2.78) | 0.34 | 2.78(2.77) | <0.7 |
| 1182.548 | 28.273 | 0.18 | 1.72(1.76) | 0.47 | 1.92(2.00) | 0.32 | 1.86(1.82) | 0.06 | 1.81(1.87) | 0.763350 |
| 1186.53 | 22.39375 | 0.78 | 2.36(2.35) | 0.91 | 2.61(2.63) | 0.7 | 2.62(2.68) | 0.5 | 2.54(2.67) | <0.7 |
| 1187.358 | 35.68919 | 0.71 | 2.61(2.53) | 0.61 | 2.80(2.87) | 0.93 | 2.96(2.99) | 0.82 | 2.95(3.01) | 0.752054 |
| 1191.517 | 36.17672 | 0.54 | 2.10(2.12) | 0.45 | 2.26(2.25) | 0.71 | 2.36(2.39) | 0.58 | 2.37(2.35) | 0.776003 |
| 1199.576 | 21.9516 | 0.83 | 2.33(2.35) | 0.8 | 2.40(2.42) | 0.79 | 2.56(2.58) | 0.58 | 2.32(2.45) | <0.7 |
| 1204.597 | 21.94117 | 0.51 | 2.14(2.15) | 0.35 | 2.11(2.09) | 0.4 | 2.18(2.21) | 0.24 | 2.01(1.92) | <0.7 |
| 1211.542 | 25.82128 | 0.42 | 1.93(1.97) | 0.38 | 2.01(2.04) | 0.45 | 2.19(2.18) | 0.32 | 2.12(2.18) | <0.7 |
| 1216.537 | 24.24347 | 0.81 | 2.72(2.72) | 0.91 | 2.96(2.97) | 0.79 | 3.02(3.11) | 0.86 | 2.96(3.05) | 0.745636 |
| 1217.529 | 35.78057 | 0.5 | 3.04(3.17) | 0.57 | 3.13(3.17) | 0.52 | 3.19(3.28) | 0.46 | 3.41(3.46) | 0.724103 |
| 1234.563 | 27.37202 | 0.93 | 2.72(2.67) | 0.91 | 2.93(2.93) | 0.84 | 3.02(2.99) | 1 | 3.02(3.09) | 0.729263 |
| 1247.523 | 22.00076 | 0.94 | 2.71(2.69) | 0.91 | 2.84(2.92) | 0.87 | 3.02(3.03) | 0.66 | 2.96(3.03) | <0.7 |
| 1262.465 | 38.233 | 0.19 | 1.88(1.76) | 0.32 | 2.13(2.10) | 0.28 | 2.08(2.07) | 0.1 | 2.23(2.43) | <0.7 |
| 1263.543 | 22.72857 | 0.61 | 2.34(2.37) | 0.77 | 2.43(2.41) | 0.69 | 2.62(2.68) | 0.42 | 2.29(2.33) | 0.710601 |
| 1265.589 | 27.08673 | 0.83 | 3.13(3.15) | 0.84 | 3.44(3.48) | 0.77 | 3.50(3.58) | 0.66 | 3.22(3.29) | 0.803325 |
| 1270.548 | 29.38075 | 0.19 | 1.97(1.89) | 0.41 | 2.13(2.09) | 0.34 | 2.04(2.01) | 0.14 | 2.22(2.23) | 0.760335 |
| 1281.585 | 27.09018 | 0.58 | 2.22(2.29) | 0.55 | 2.24(2.25) | 0.53 | 2.40(2.43) | 0.28 | 2.37(2.47) | <0.7 |
| 1283.554 | 27.26645 | 0.22 | 1.97(1.99) | 0.49 | 2.22(2.25) | 0.32 | 2.24(2.22) | 0.36 | 2.11(2.18) | 0.706722 |
| 1286.543 | 29.40513 | 0.25 | 1.74(1.71) | 0.46 | 2.05(2.06) | 0.31 | 1.96(1.92) | 0.14 | 2.11(2.10) | 0.702782 |
| 1295.356 | 34.16189 | 0.26 | 2.04(2.05) | 0.26 | 2.45(2.44) | 0.51 | 2.31(2.33) | 0.16 | 2.12(2.14) | <0.7 |
| 1297.582 | 27.36504 | 0.73 | 2.99(3.05) | 0.65 | 3.09(3.17) | 0.63 | 3.12(3.16) | 0.34 | 2.90(3.13) | <0.7 |
| 1299.583 | 22.38183 | 0.73 | 2.49(2.50) | 0.75 | 2.52(2.54) | 0.66 | 2.51(2.57) | 0.22 | 2.26(2.29) | 0.780177 |
| 1324.592 | 28.70152 | 0.65 | 2.45(2.45) | 0.37 | 2.41(2.45) | 0.39 | 2.44(2.46) | 0.54 | 2.71(2.79) | <0.7 |
| 1326.55 | 29.20429 | 0.46 | 1.92(1.94) | 0.7 | 2.31(2.35) | 0.47 | 2.19(2.16) | 0.32 | 2.05(2.00) | <0.7 |
| 1337.62 | 38.19948 | 0.46 | 1.96(1.92) | 0.39 | 2.00(1.99) | 0.4 | 2.11(2.07) | 0.24 | 2.06(2.09) | <0.7 |
| 1339.602 | 27.48587 | 0.55 | 2.00(2.04) | 0.75 | 2.16(2.19) | 0.59 | 2.17(2.16) | 0.4 | 2.14(2.20) | 0.764926 |
| 1351.635 | 38.75677 | 0.61 | 2.24(2.29) | 0.54 | 2.14(2.09) | 0.54 | 2.31(2.35) | 0.46 | 2.30(2.27) | 0.715405 |
| 1352.556 | 29.76547 | 0.43 | 1.86(1.88) | 0.68 | 2.16(2.17) | 0.42 | 2.00(1.92) | 0.38 | 2.04(2.14) | <0.7 |
| 1352.779 | 24.60145 | 0.62 | 2.38(2.34) | 0.38 | 2.54(2.54) | 0.54 | 2.31(2.30) | 0.1 | 2.33(2.29) | <0.7 |
| 1353.588 | 21.48296 | 0.69 | 2.39(2.44) | 0.55 | 2.34(2.29) | 0.54 | 2.57(2.62) | 0.44 | 2.59(2.60) | <0.7 |
| 1353.656 | 25.63162 | 0.74 | 2.27(2.27) | 0.73 | 2.45(2.52) | 0.81 | 2.41(2.41) | 0.7 | 2.46(2.55) | 0.815811 |
| 1357.578 | 30.02141 | 0.35 | 1.81(1.82) | 0.52 | 2.03(2.02) | 0.38 | 1.91(1.96) | 0.46 | 2.07(2.12) | <0.7 |
| 1358.38 | 36.46108 | 0.96 | 3.05(3.07) | 1 | 3.28(3.33) | 0.94 | 3.22(3.22) | 0.86 | 3.09(3.13) | 0.718347 |
| 1367.643 | 38.88257 | 0.85 | 2.63(2.64) | 0.83 | 2.51(2.58) | 0.73 | 2.68(2.74) | 0.68 | 2.77(2.74) | 0.738423 |
| 1378.613 | 28.822 | 0.97 | 3.17(3.17) | 0.99 | 3.36(3.39) | 0.89 | 3.20(3.26) | 0.92 | 3.20(3.25) | 0.876530 |
| 1383.593 | 27.62604 | 0.81 | 2.25(2.31) | 0.83 | 2.31(2.31) | 0.51 | 2.36(2.41) | 0.6 | 2.26(2.36) | <0.7 |
| 1405.635 | 20.13912 | 0.39 | 2.04(2.10) | 0.5 | 2.23(2.26) | 0.29 | 2.11(2.04) | 0.12 | 2.17(2.26) | <0.7 |
| 1406.635 | 28.15559 | 0.42 | 2.08(2.01) | 0.66 | 2.35(2.38) | 0.37 | 2.21(2.22) | 0.38 | 2.14(2.07) | <0.7 |
| 1407.657 | 37.22917 | 0.47 | 2.11(2.16) | 0.74 | 2.31(2.35) | 0.5 | 2.33(2.35) | 0.32 | 2.21(2.22) | 0.704479 |
| 1413.55 | 25.55998 | 0.52 | 2.31(2.26) | 0.55 | 2.50(2.42) | 0.48 | 2.29(2.25) | 0.52 | 2.64(2.67) | <0.7 |
| 1422.597 | 21.72293 | 0.98 | 3.40(3.46) | 0.96 | 3.34(3.40) | 0.83 | 3.37(3.45) | 0.86 | 3.32(3.43) | <0.7 |
| 1422.677 | 28.14366 | 0.82 | 3.38(3.45) | 0.87 | 3.33(3.37) | 0.71 | 3.26(3.35) | 0.52 | 3.41(3.42) | <0.7 |
| 1425.587 | 22.31776 | 0.96 | 3.32(3.32) | 0.97 | 3.45(3.45) | 0.93 | 3.32(3.29) | 0.86 | 3.16(3.23) | 0.719410 |
| 1435.703 | 22.54087 | 0.81 | 2.51(2.53) | 0.64 | 2.41(2.40) | 0.58 | 2.39(2.42) | 0.34 | 2.57(2.53) | <0.7 |
| 1438.667 | 27.87549 | 0.95 | 3.41(3.55) | 0.95 | 3.45(3.54) | 0.89 | 3.38(3.46) | 0.78 | 3.18(3.27) | 0.711032 |
| 1440.562 | 24.30044 | 0.61 | 2.34(2.33) | 0.66 | 2.39(2.44) | 0.47 | 2.34(2.39) | 0.22 | 2.24(2.32) | 0.708437 |
| 1449.641 | 21.85606 | 1 | 3.57(3.57) | 0.98 | 3.64(3.65) | 0.94 | 3.50(3.51) | 0.94 | 3.40(3.50) | <0.7 |
| 1451.693 | 22.55358 | 0.94 | 3.01(3.04) | 0.86 | 2.94(2.94) | 0.89 | 2.96(2.97) | 0.78 | 2.83(2.91) | 0.706490 |
| 1460.804 | 22.76516 | 0.11 | 2.52(2.68) | 0.12 | 3.27(3.01) | 0.11 | 2.74(2.75) | 0.46 | 3.80(3.63) | 0.718643 |
| 1466.653 | 28.51877 | 0.35 | 2.17(2.13) | 0.63 | 2.48(2.66) | 0.44 | 2.27(2.15) | 0.5 | 2.26(2.34) | <0.7 |
| 1467.795 | 23.8873 | 0.33 | 2.76(2.56) | 0.29 | 2.98(2.96) | 0.36 | 3.00(3.11) | 0.48 | 2.81(2.79) | <0.7 |
| 1467.807 | 24.68522 | 0.82 | 3.10(3.17) | 0.69 | 2.91(2.95) | 0.74 | 3.06(3.08) | 0.54 | 2.59(2.59) | <0.7 |
| 1469.668 | 23.69358 | 0.97 | 3.38(3.43) | 0.98 | 3.32(3.38) | 0.95 | 3.28(3.29) | 0.9 | 3.21(3.25) | 0.724425 |
| 1482.666 | 22.46624 | 0.59 | 2.46(2.48) | 0.38 | 2.56(2.60) | 0.52 | 2.53(2.58) | 0.2 | 2.22(2.14) | <0.7 |
| 1485.674 | 23.76807 | 0.93 | 3.03(3.05) | 0.96 | 3.05(3.07) | 0.88 | 2.89(2.98) | 0.88 | 2.81(2.87) | <0.7 |
| 1486.683 | 21.15232 | 0.69 | 2.19(2.17) | 0.62 | 2.35(2.42) | 0.66 | 2.33(2.38) | 0.46 | 2.31(2.34) | 0.712253 |
| 1487.652 | 29.62173 | 0.53 | 2.31(2.34) | 0.82 | 2.70(2.75) | 0.53 | 2.51(2.52) | 0.36 | 2.31(2.27) | 0.783325 |
| 1491.739 | 39.83392 | 0.66 | 2.44(2.53) | 0.61 | 2.31(2.35) | 0.53 | 2.35(2.41) | 0.42 | 2.44(2.42) | <0.7 |
| 1496.684 | 30.37452 | 0.31 | 1.78(1.73) | 0.5 | 2.19(2.20) | 0.29 | 2.06(2.05) | 0.12 | 2.14(2.19) | 0.731998 |
| 1508.678 | 29.33272 | 0.91 | 3.29(3.34) | 0.94 | 3.34(3.42) | 0.81 | 3.22(3.31) | 0.82 | 3.15(3.07) | 0.701449 |
| 1510.654 | 28.27854 | 0.34 | 2.28(2.33) | 0.3 | 2.13(2.11) | 0.25 | 2.43(2.42) | 0.56 | 2.65(2.72) | <0.7 |
| 1510.682 | 20.16625 | 0.75 | 2.51(2.54) | 0.45 | 2.65(2.73) | 0.54 | 2.41(2.47) | 0.36 | 2.46(2.44) | <0.7 |
| 1521.688 | 30.52821 | 0.41 | 1.93(1.83) | 0.69 | 2.26(2.20) | 0.25 | 2.06(2.09) | 0.28 | 1.99(1.89) | <0.7 |
| 1522.712 | 22.89534 | 0.65 | 2.53(2.52) | 0.43 | 2.29(2.30) | 0.35 | 2.31(2.38) | 0.4 | 2.36(2.38) | <0.7 |
| 1523.841 | 29.75377 | 0.25 | 2.66(2.70) | 0.44 | 3.02(3.02) | 0.34 | 2.79(2.75) | 0.38 | 2.93(2.97) | <0.7 |
| 1524.654 | 20.02625 | 0.83 | 2.49(2.50) | 0.87 | 2.71(2.74) | 0.74 | 2.45(2.47) | 0.58 | 2.47(2.62) | <0.7 |
| 1526.69 | 23.91861 | 0.55 | 1.96(1.97) | 0.55 | 2.13(2.17) | 0.51 | 2.05(2.05) | 0.44 | 2.04(2.24) | <0.7 |
| 1542.692 | 23.96014 | 0.54 | 2.11(2.13) | 0.49 | 2.31(2.35) | 0.38 | 2.18(2.21) | 0.28 | 2.10(2.15) | <0.7 |
| 1549.696 | 39.48611 | 0.64 | 2.53(2.51) | 0.61 | 2.26(2.23) | 0.55 | 2.41(2.46) | 0.46 | 2.34(2.35) | <0.7 |
| 1567.702 | 20.19208 | 0.94 | 2.89(2.91) | 0.78 | 2.75(2.89) | 0.71 | 2.81(2.79) | 0.46 | 2.78(2.87) | 0.725436 |
| 1576.6 | 26.37432 | 0.92 | 3.07(3.08) | 0.99 | 3.29(3.32) | 0.71 | 3.13(3.17) | 0.78 | 2.96(3.01) | <0.7 |
| 1576.743 | 19.50785 | 0.69 | 2.40(2.40) | 0.68 | 2.46(2.49) | 0.6 | 2.55(2.60) | 0.44 | 2.25(2.29) | <0.7 |
| 1580.886 | 24.84996 | 0.7 | 3.23(3.31) | 0.5 | 2.99(3.13) | 0.59 | 3.11(3.10) | 0.48 | 2.52(2.42) | <0.7 |
| 1583.701 | 23.26777 | 0.84 | 2.62(2.63) | 0.8 | 2.65(2.67) | 0.72 | 2.47(2.52) | 0.48 | 2.27(2.27) | 0.778938 |
| 1586.738 | 28.88186 | 0.76 | 2.28(2.32) | 0.77 | 2.32(2.35) | 0.65 | 2.27(2.32) | 0.56 | 2.25(2.31) | <0.7 |
| 1588.706 | 30.15033 | 0.86 | 2.43(2.47) | 0.82 | 2.83(2.87) | 0.63 | 2.55(2.62) | 0.26 | 2.43(2.49) | <0.7 |
| 1592.697 | 22.1803 | 0.7 | 2.62(2.66) | 0.8 | 2.63(2.59) | 0.38 | 2.52(2.46) | 0.34 | 2.39(2.38) | <0.7 |
| 1594.726 | 23.05294 | 0.84 | 2.61(2.63) | 0.79 | 2.58(2.61) | 0.8 | 2.54(2.55) | 0.52 | 2.39(2.34) | <0.7 |
| 1594.762 | 40.21545 | 0.86 | 3.11(3.19) | 0.89 | 2.84(2.92) | 0.73 | 2.89(2.92) | 0.54 | 2.91(2.98) | 0.753628 |
| 1595.704 | 30.00662 | 0.92 | 2.65(2.67) | 0.91 | 2.64(2.67) | 0.71 | 2.59(2.62) | 0.66 | 2.61(2.61) | <0.7 |
| 1608.684 | 22.34901 | 0.65 | 2.30(2.29) | 0.76 | 2.36(2.43) | 0.49 | 2.21(2.24) | 0.26 | 1.96(2.01) | 0.716195 |
| 1608.73 | 30.93209 | 0.65 | 2.43(2.36) | 0.88 | 2.80(2.81) | 0.45 | 2.25(2.21) | 0.48 | 2.25(2.30) | <0.7 |
| 1623.727 | 24.12394 | 0.99 | 3.80(3.80) | 0.98 | 3.73(3.75) | 0.99 | 3.64(3.71) | 0.92 | 3.64(3.67) | <0.7 |
| 1624.546 | 37.72626 | 0.95 | 2.87(2.93) | 0.98 | 3.15(3.23) | 0.91 | 3.01(2.99) | 0.68 | 2.85(2.96) | <0.7 |
| 1627.695 | 29.45318 | 0.88 | 2.56(2.57) | 0.68 | 2.54(2.56) | 0.77 | 2.56(2.56) | 0.6 | 2.51(2.56) | <0.7 |
| 1634.799 | 29.71747 | 0.81 | 2.69(2.73) | 0.72 | 2.76(2.76) | 0.74 | 2.68(2.68) | 0.66 | 2.77(2.82) | <0.7 |
| 1635.76 | 30.33571 | 0.81 | 3.07(3.18) | 0.74 | 3.17(3.29) | 0.56 | 3.07(3.21) | 0.58 | 2.89(3.11) | <0.7 |
| 1636.699 | 20.03245 | 0.88 | 2.79(2.81) | 0.89 | 2.96(2.98) | 0.79 | 2.80(2.81) | 0.74 | 2.63(2.63) | <0.7 |
| 1636.741 | 30.25104 | 0.27 | 3.07(3.16) | 0.4 | 3.24(3.28) | 0.36 | 3.08(3.16) | 0.46 | 3.21(3.28) | <0.7 |
| 1638.728 | 20.22988 | 0.9 | 3.03(3.10) | 0.71 | 2.91(3.00) | 0.74 | 2.83(2.91) | 0.66 | 2.68(2.80) | 0.723890 |
| 1640.581 | 23.24178 | 0.97 | 3.44(3.47) | 0.94 | 3.62(3.66) | 0.78 | 3.53(3.57) | 0.72 | 3.28(3.32) | <0.7 |
| 1640.678 | 28.04219 | 0.58 | 2.04(2.06) | 0.85 | 2.36(2.37) | 0.46 | 2.13(2.22) | 0.44 | 2.01(2.10) | <0.7 |
| 1651.79 | 40.66166 | 0.93 | 3.65(3.66) | 0.94 | 3.30(3.34) | 0.83 | 3.40(3.41) | 0.72 | 3.57(3.56) | 0.714100 |
| 1664.746 | 29.81371 | 0.89 | 2.77(2.78) | 0.8 | 2.77(2.79) | 0.71 | 2.59(2.68) | 0.74 | 2.79(2.84) | <0.7 |
| 1666.775 | 30.66426 | 0.5 | 2.29(2.35) | 0.64 | 2.35(2.40) | 0.43 | 2.26(2.29) | 0.5 | 2.28(2.25) | 0.720815 |
| 1668.805 | 40.46556 | 0.3 | 2.91(2.91) | 0.27 | 2.60(2.65) | 0.33 | 2.84(2.85) | 0.32 | 2.69(2.76) | <0.7 |
| 1669.689 | 21.46473 | 0.85 | 2.53(2.54) | 0.76 | 2.49(2.52) | 0.69 | 2.45(2.40) | 0.54 | 2.55(2.38) | <0.7 |
| 1671.539 | 37.6477 | 0.29 | 1.89(1.87) | 0.51 | 2.07(2.09) | 0.49 | 1.97(1.99) | 0.22 | 2.09(2.10) | <0.7 |
| 1679.699 | 22.60456 | 0.53 | 2.27(2.28) | 0.39 | 2.37(2.41) | 0.28 | 2.22(2.25) | 0.18 | 2.12(2.04) | 0.703127 |
| 1680.752 | 30.02747 | 0.95 | 3.38(3.43) | 0.88 | 3.40(3.44) | 0.74 | 3.32(3.35) | 0.82 | 3.24(3.40) | <0.7 |
| 1692.798 | 30.88753 | 0.88 | 2.67(2.72) | 0.88 | 2.86(2.94) | 0.74 | 2.55(2.52) | 0.74 | 2.51(2.58) | 0.748121 |
| 1693.762 | 20.50581 | 0.73 | 2.24(2.26) | 0.73 | 2.28(2.27) | 0.52 | 2.13(2.10) | 0.3 | 2.20(2.16) | <0.7 |
| 1696.718 | 23.94985 | 0.52 | 2.22(2.29) | 0.35 | 2.27(2.31) | 0.43 | 2.22(2.27) | 0.28 | 2.18(2.34) | <0.7 |
| 1706.777 | 22.68944 | 0.99 | 2.87(2.86) | 0.97 | 2.82(2.83) | 0.95 | 2.71(2.73) | 0.76 | 2.56(2.62) | <0.7 |
| 1708.785 | 30.59054 | 0.9 | 2.69(2.63) | 0.97 | 2.97(2.97) | 0.75 | 2.65(2.67) | 0.72 | 2.58(2.63) | <0.7 |
| 1714.548 | 37.84911 | 0.42 | 2.01(1.99) | 0.65 | 2.22(2.22) | 0.55 | 2.13(2.18) | 0.38 | 2.16(2.23) | <0.7 |
| 1716.657 | 20.17727 | 0.59 | 2.32(2.32) | 0.63 | 2.48(2.54) | 0.57 | 2.40(2.41) | 0.18 | 2.13(2.18) | <0.7 |
| 1725.591 | 38.32196 | 0.89 | 2.98(3.04) | 0.98 | 3.31(3.36) | 0.91 | 3.05(3.05) | 0.72 | 2.85(3.00) | <0.7 |
| 1728.771 | 36.767 | 0.81 | 2.44(2.46) | 0.83 | 2.51(2.52) | 0.7 | 2.32(2.36) | 0.36 | 2.43(2.46) | 0.727168 |
| 1731.746 | 22.72137 | 0.88 | 2.40(2.43) | 0.82 | 2.29(2.31) | 0.65 | 2.26(2.31) | 0.42 | 2.26(2.29) | 0.714774 |
| 1732.771 | 28.17527 | 0.63 | 3.31(3.35) | 0.9 | 3.37(3.41) | 0.83 | 3.30(3.29) | 0.74 | 3.28(3.31) | <0.7 |
| 1741.737 | 30.19875 | 0.58 | 2.23(2.24) | 0.49 | 2.10(2.12) | 0.4 | 2.09(2.13) | 0.32 | 2.12(2.20) | <0.7 |
| 1750.784 | 23.8278 | 0.85 | 2.77(2.78) | 0.92 | 2.85(2.88) | 0.87 | 2.90(2.93) | 0.68 | 2.92(2.93) | <0.7 |
| 1754.895 | 31.25885 | 0.84 | 3.54(3.56) | 0.86 | 3.70(3.72) | 0.82 | 3.60(3.62) | 0.54 | 3.37(3.42) | <0.7 |
| 1764.68 | 19.90654 | 0.3 | 2.15(2.04) | 0.51 | 2.38(2.34) | 0.48 | 2.38(2.45) | 0.16 | 2.28(2.13) | <0.7 |
| 1764.79 | 29.82516 | 0.55 | 2.32(2.22) | 0.47 | 2.29(2.14) | 0.26 | 2.19(2.06) | 0.28 | 2.22(2.15) | <0.7 |
| 1764.809 | 26.55983 | 0.65 | 2.70(2.86) | 0.37 | 2.59(2.77) | 0.5 | 2.59(2.65) | 0.58 | 2.49(2.61) | <0.7 |
| 1765.809 | 31.00023 | 0.81 | 3.05(3.09) | 0.83 | 3.20(3.27) | 0.83 | 3.00(3.06) | 0.84 | 3.10(3.09) | 0.705858 |
| 1767.781 | 19.81004 | 0.51 | 2.44(2.47) | 0.5 | 2.36(2.34) | 0.48 | 2.29(2.36) | 0.28 | 2.43(2.26) | <0.7 |
| 1769.713 | 28.13518 | 0.81 | 2.54(2.55) | 0.93 | 2.80(2.85) | 0.73 | 2.53(2.57) | 0.58 | 2.38(2.45) | <0.7 |
| 1770.592 | 38.25859 | 0.25 | 1.88(1.88) | 0.53 | 2.01(2.01) | 0.37 | 1.87(1.91) | 0.24 | 1.87(1.89) | <0.7 |
| 1782.842 | 25.91265 | 0.8 | 2.74(2.77) | 0.53 | 2.73(2.75) | 0.68 | 2.71(2.76) | 0.56 | 2.78(2.84) | <0.7 |
| 1786.581 | 38.29337 | 0.4 | 2.15(2.14) | 0.65 | 2.32(2.39) | 0.38 | 2.11(2.08) | 0.42 | 2.15(2.01) | <0.7 |
| 1793.884 | 32.3738 | 0.87 | 2.54(2.54) | 0.91 | 2.57(2.58) | 0.87 | 2.56(2.62) | 0.62 | 2.41(2.33) | <0.7 |
| 1794.796 | 23.91532 | 0.98 | 3.29(3.29) | 0.93 | 3.27(3.29) | 0.86 | 3.20(3.26) | 0.7 | 3.11(3.15) | <0.7 |
| 1796.772 | 30.92154 | 0.28 | 2.01(1.95) | 0.44 | 2.05(2.10) | 0.31 | 2.03(1.92) | 0.44 | 2.09(2.11) | <0.7 |
| 1798.716 | 36.94821 | 0.22 | 2.00(1.97) | 0.53 | 2.25(2.25) | 0.38 | 2.15(2.19) | 0.24 | 2.23(2.33) | 0.711874 |
| 1806.827 | 23.06136 | 0.71 | 2.29(2.34) | 0.74 | 2.27(2.24) | 0.41 | 2.22(2.22) | 0.4 | 2.27(2.24) | <0.7 |
| 1807.809 | 20.64857 | 0.96 | 3.08(3.14) | 0.89 | 3.16(3.25) | 0.92 | 3.03(3.08) | 0.8 | 2.81(2.92) | <0.7 |
| 1813.715 | 31.69499 | 0.96 | 3.26(3.28) | 0.96 | 3.53(3.55) | 0.93 | 3.26(3.31) | 0.78 | 3.01(3.14) | <0.7 |
| 1817.694 | 20.23435 | 0.96 | 3.24(3.34) | 0.95 | 3.42(3.48) | 0.86 | 3.26(3.30) | 0.7 | 3.04(3.24) | 0.726165 |
| 1818.827 | 30.95312 | 0.65 | 2.59(2.49) | 0.57 | 2.70(2.56) | 0.54 | 2.67(2.58) | 0.8 | 3.22(3.40) | <0.7 |
| 1819.796 | 23.36311 | 0.98 | 3.54(3.58) | 0.98 | 3.57(3.56) | 0.9 | 3.49(3.50) | 0.96 | 3.42(3.44) | <0.7 |
| 1821.815 | 30.17125 | 0.71 | 2.41(2.40) | 0.69 | 2.40(2.39) | 0.49 | 2.39(2.37) | 0.56 | 2.12(2.11) | <0.7 |
| 1822.735 | 30.87417 | 0.65 | 2.50(2.49) | 0.61 | 2.50(2.52) | 0.46 | 2.41(2.44) | 0.4 | 2.27(2.34) | <0.7 |
| 1822.829 | 26.99979 | 0.64 | 2.25(2.25) | 0.31 | 2.29(2.31) | 0.45 | 2.35(2.40) | 0.42 | 2.40(2.39) | <0.7 |
| 1823.988 | 24.39771 | 0.76 | 2.91(2.84) | 0.64 | 2.96(3.00) | 0.79 | 3.02(3.06) | 0.46 | 2.52(2.42) | <0.7 |
| 1825.786 | 20.13497 | 0.97 | 3.27(3.29) | 0.96 | 3.35(3.40) | 0.89 | 3.09(3.13) | 0.76 | 2.84(2.93) | 0.703104 |
| 1825.796 | 31.93096 | 0.5 | 2.13(2.16) | 0.6 | 2.10(2.11) | 0.68 | 2.29(2.31) | 0.46 | 2.16(2.21) | <0.7 |
| 1828.848 | 21.20199 | 0.5 | 2.35(2.37) | 0.34 | 2.18(2.15) | 0.42 | 2.38(2.48) | 0.34 | 2.40(2.36) | <0.7 |
| 1835.709 | 19.91313 | 0.71 | 2.65(2.74) | 0.83 | 2.89(2.95) | 0.69 | 2.71(2.69) | 0.38 | 2.45(2.46) | <0.7 |
| 1837.8 | 30.55694 | 0.31 | 2.32(2.24) | 0.43 | 2.48(2.47) | 0.37 | 2.32(2.38) | 0.34 | 2.13(2.16) | <0.7 |
| 1838.818 | 27.0581 | 0.63 | 2.23(2.25) | 0.4 | 2.13(2.23) | 0.37 | 2.32(2.35) | 0.28 | 2.41(2.45) | <0.7 |
| 1840.836 | 41.17953 | 0.24 | 2.15(2.26) | 0.29 | 1.99(1.96) | 0.25 | 2.14(2.12) | 0.32 | 2.18(2.26) | 0.711541 |
| 1841.751 | 35.65976 | 0.32 | 1.83(1.90) | 0.65 | 2.23(2.27) | 0.34 | 1.90(1.98) | 0.22 | 1.91(1.90) | 0.725559 |
| 1843.779 | 30.63276 | 0.03 | 2.07(2.19) | 0.13 | 1.83(1.66) | 0.1 | 2.01(1.91) | 0.6 | 2.55(2.39) | 0.803894 |
| 1848.806 | 30.80986 | 0.77 | 2.16(2.20) | 0.68 | 2.11(2.12) | 0.59 | 2.13(2.14) | 0.52 | 2.04(2.12) | <0.7 |
| 1858.839 | 24.2646 | 0.27 | 2.50(2.57) | 0.48 | 2.76(2.61) | 0.35 | 2.62(2.68) | 0.32 | 2.86(2.82) | <0.7 |
| 1859.828 | 24.41139 | 0.55 | 2.60(2.65) | 0.34 | 2.47(2.56) | 0.46 | 2.51(2.56) | 0.2 | 2.09(2.11) | <0.7 |
| 1860.461 | 34.21703 | 0.51 | 2.43(2.46) | 0.82 | 2.56(2.65) | 0.52 | 2.60(2.61) | 0.42 | 2.42(2.36) | <0.7 |
| 1860.826 | 21.40014 | 0.79 | 2.83(2.85) | 0.76 | 2.88(2.93) | 0.71 | 2.84(2.95) | 0.32 | 2.47(2.45) | 0.779646 |
| 1865.808 | 32.98199 | 0.35 | 1.88(1.94) | 0.39 | 2.04(1.95) | 0.45 | 1.91(1.93) | 0.44 | 1.95(1.91) | 0.711935 |
| 1874.831 | 30.82379 | 0.5 | 2.23(2.22) | 0.56 | 2.24(2.24) | 0.45 | 2.10(2.09) | 0.34 | 2.01(2.04) | <0.7 |
| 1876.866 | 22.20126 | 0.96 | 3.20(3.24) | 0.94 | 3.01(3.05) | 0.92 | 3.13(3.19) | 0.9 | 2.99(3.08) | <0.7 |
| 1876.842 | 23.38023 | 0.78 | 2.43(2.48) | 0.83 | 2.43(2.43) | 0.58 | 2.34(2.36) | 0.46 | 2.42(2.40) | <0.7 |
| 1878.59 | 30.77853 | 0.47 | 2.47(2.44) | 0.69 | 2.79(2.85) | 0.45 | 2.74(2.83) | 0.5 | 2.81(2.79) | <0.7 |
| 1878.792 | 20.7224 | 0.71 | 2.38(2.37) | 0.81 | 2.55(2.62) | 0.57 | 2.42(2.41) | 0.38 | 2.49(2.45) | <0.7 |
| 1880.895 | 43.9095 | 0.27 | 2.52(2.63) | 0.28 | 2.60(2.70) | 0.28 | 2.61(2.75) | 0.36 | 2.57(2.68) | 0.731240 |
| 1885.651 | 38.81996 | 0.49 | 2.09(2.11) | 0.72 | 2.24(2.22) | 0.59 | 2.10(2.13) | 0.42 | 2.10(2.14) | <0.7 |
| 1889.868 | 33.0683 | 0.54 | 2.35(2.42) | 0.2 | 2.18(2.23) | 0.42 | 2.22(2.25) | 0.28 | 1.93(2.04) | <0.7 |
| 1892.869 | 22.19629 | 0.93 | 2.77(2.83) | 0.85 | 2.54(2.56) | 0.82 | 2.53(2.63) | 0.72 | 2.55(2.57) | <0.7 |
| 1892.973 | 24.55813 | 0.68 | 2.54(2.59) | 0.47 | 2.64(2.73) | 0.64 | 2.63(2.69) | 0.34 | 2.32(2.39) | 0.712271 |
| 1915.909 | 31.29872 | 0.16 | 1.66(1.64) | 0.31 | 2.02(2.07) | 0.18 | 2.06(2.03) | 0.24 | 1.98(2.02) | 0.712192 |
| 1916.768 | 20.32244 | 0.87 | 2.90(2.97) | 0.87 | 3.11(3.24) | 0.78 | 2.93(2.98) | 0.66 | 2.69(2.81) | <0.7 |
| 1916.946 | 34.19299 | 0.59 | 2.04(2.07) | 0.71 | 2.14(2.16) | 0.42 | 1.92(2.00) | 0.36 | 1.89(1.93) | <0.7 |
| 1925.815 | 23.1958 | 0.47 | 2.04(2.00) | 0.76 | 2.32(2.34) | 0.41 | 2.07(2.12) | 0.16 | 1.97(2.02) | 0.726988 |
| 1933.877 | 21.62452 | 0.97 | 2.84(2.87) | 0.89 | 2.61(2.63) | 0.89 | 2.67(2.66) | 0.86 | 2.58(2.57) | <0.7 |
| 1934.786 | 19.94225 | 0.59 | 2.52(2.50) | 0.75 | 2.71(2.73) | 0.56 | 2.49(2.46) | 0.28 | 2.26(2.26) | <0.7 |
| 1938.881 | 21.38561 | 0.61 | 2.41(2.35) | 0.57 | 2.19(2.21) | 0.5 | 2.31(2.29) | 0.28 | 2.00(2.12) | 0.726549 |
| 1942.837 | 30.96243 | 0.65 | 2.08(2.07) | 0.55 | 2.02(2.03) | 0.54 | 2.07(2.17) | 0.34 | 2.02(2.07) | <0.7 |
| 1943.007 | 24.9416 | 0.22 | 3.07(3.07) | 0.11 | 2.77(2.68) | 0.21 | 3.13(3.20) | 0.6 | 3.81(4.01) | 0.721416 |
| 1945.004 | 33.70855 | 0.88 | 2.31(2.35) | 0.83 | 2.37(2.43) | 0.78 | 2.32(2.34) | 0.62 | 2.20(2.24) | <0.7 |
| 1949.889 | 21.65736 | 0.78 | 2.49(2.47) | 0.75 | 2.28(2.26) | 0.71 | 2.56(2.53) | 0.54 | 2.56(2.51) | <0.7 |
| 1955.882 | 28.11064 | 0.44 | 2.31(2.41) | 0.78 | 2.66(2.68) | 0.42 | 2.41(2.51) | 0.32 | 2.27(2.33) | 0.747300 |
| 1959.006 | 25.01828 | 0.11 | 2.84(2.74) | 0.06 | 2.83(3.10) | 0.15 | 2.37(2.25) | 0.64 | 3.25(3.43) | 0.767257 |
| 1963.88 | 31.74253 | 0.59 | 2.27(2.29) | 0.51 | 2.33(2.37) | 0.39 | 2.29(2.24) | 0.28 | 2.00(2.16) | <0.7 |
| 1969.838 | 25.22952 | 0.73 | 2.69(2.79) | 0.88 | 3.06(3.13) | 0.68 | 2.74(2.72) | 0.42 | 2.53(2.50) | <0.7 |
| 1976.882 | 32.38465 | 0.37 | 2.37(2.43) | 0.65 | 2.46(2.53) | 0.5 | 2.35(2.45) | 0.3 | 2.18(2.26) | <0.7 |
| 1977.918 | 32.19436 | 0.6 | 2.57(2.53) | 0.34 | 2.48(2.56) | 0.44 | 2.44(2.54) | 0.7 | 2.44(2.56) | <0.7 |
| 1991.941 | 22.04607 | 0.96 | 3.04(3.07) | 0.86 | 2.75(2.82) | 0.9 | 2.86(2.95) | 0.7 | 2.69(2.68) | 0.723429 |
| 1996.786 | 20.9757 | 0.67 | 2.51(2.47) | 0.83 | 2.76(2.79) | 0.54 | 2.73(2.79) | 0.36 | 2.44(2.42) | <0.7 |
| 2003.939 | 24.61864 | 0.27 | 2.00(1.97) | 0.25 | 2.07(2.09) | 0.32 | 1.85(1.88) | 0.44 | 2.24(2.23) | <0.7 |
| 2007.945 | 22.10222 | 0.96 | 3.22(3.24) | 0.92 | 2.99(2.98) | 0.9 | 3.21(3.32) | 0.8 | 3.01(3.04) | <0.7 |
| 2008.902 | 32.2865 | 0.27 | 2.63(2.67) | 0.5 | 2.75(2.72) | 0.21 | 2.69(2.76) | 0.42 | 2.67(2.75) | <0.7 |
| 2013.893 | 31.75577 | 0.33 | 1.95(1.96) | 0.52 | 1.97(2.04) | 0.51 | 1.94(1.92) | 0.36 | 1.99(2.05) | <0.7 |
| 2016.044 | 21.3587 | 0.15 | 2.43(2.30) | 0.02 | 2.27(1.88) | 0.19 | 2.28(2.15) | 0.56 | 3.10(3.24) | 0.728142 |
| 2023.908 | 21.48129 | 0.75 | 2.38(2.36) | 0.69 | 2.26(2.27) | 0.66 | 2.42(2.44) | 0.58 | 2.32(2.33) | <0.7 |
| 2029.853 | 20.39044 | 0.65 | 2.35(2.31) | 0.66 | 2.49(2.53) | 0.52 | 2.40(2.46) | 0.22 | 2.00(2.07) | 0.752153 |
| 2030.93 | 32.60772 | 0.94 | 2.95(2.98) | 0.94 | 2.97(2.98) | 0.91 | 2.92(2.93) | 0.82 | 2.84(2.94) | <0.7 |
| 2034.995 | 40.18541 | 0.35 | 2.01(2.06) | 0.59 | 2.16(2.22) | 0.28 | 2.02(2.08) | 0.24 | 2.12(2.11) | <0.7 |
| 2039.129 | 21.77864 | 0.75 | 3.12(3.17) | 0.77 | 3.19(3.23) | 0.83 | 3.18(3.19) | 0.48 | 2.78(2.87) | <0.7 |
| 2041.979 | 28.49748 | 0.35 | 1.84(1.91) | 0.65 | 1.94(1.97) | 0.21 | 1.93(1.96) | 0.28 | 1.80(1.83) | <0.7 |
| 2042.071 | 25.1431 | 0.26 | 3.07(3.19) | 0.15 | 2.69(2.55) | 0.3 | 2.80(2.87) | 0.52 | 3.77(3.76) | 0.700590 |
| 2048.927 | 24.46324 | 0.98 | 3.21(3.22) | 0.97 | 3.02(3.02) | 0.9 | 3.07(3.10) | 0.94 | 3.30(3.30) | <0.7 |
| 2055.939 | 25.43797 | 0.94 | 2.90(2.96) | 0.94 | 2.84(2.87) | 0.94 | 2.89(2.94) | 0.72 | 3.02(3.07) | <0.7 |
| 2058.937 | 23.15082 | 0.65 | 2.26(2.33) | 0.71 | 2.31(2.31) | 0.68 | 2.40(2.47) | 0.38 | 2.34(2.36) | <0.7 |
| 2063.932 | 21.94861 | 1 | 3.76(3.79) | 0.99 | 3.60(3.66) | 0.99 | 3.62(3.65) | 0.96 | 3.45(3.51) | 0.759410 |
| 2064.918 | 24.45992 | 0.96 | 2.77(2.84) | 0.86 | 2.55(2.55) | 0.83 | 2.65(2.66) | 0.86 | 2.80(2.85) | <0.7 |
| 2067.818 | 20.62077 | 0.92 | 2.83(2.90) | 0.92 | 3.01(3.07) | 0.8 | 2.93(2.95) | 0.52 | 2.04(2.75) | 0.745929 |
| 2076.945 | 21.77894 | 0.56 | 2.59(2.64) | 0.49 | 2.75(2.75) | 0.55 | 2.83(2.94) | 0.5 | 2.76(2.76) | <0.7 |
| 2087.966 | 32.91176 | 0.97 | 3.12(3.19) | 0.91 | 3.10(3.12) | 0.96 | 3.07(3.12) | 0.84 | 2.95(2.95) | 0.728850 |
| 2088.875 | 23.6675 | 0.44 | 1.97(1.97) | 0.76 | 2.20(2.26) | 0.42 | 2.07(2.07) | 0.12 | 2.02(2.10) | 0.714236 |
| 2089.958 | 39.51667 | 0.27 | 2.11(2.16) | 0.53 | 2.44(2.42) | 0.21 | 2.08(2.03) | 0.2 | 2.21(2.23) | <0.7 |
| 2109.923 | 24.06903 | 0.26 | 2.33(2.41) | 0.43 | 2.29(2.31) | 0.37 | 2.25(2.30) | 0.26 | 2.19(2.18) | <0.7 |
| 2117.934 | 32.96822 | 0.35 | 1.99(2.02) | 0.37 | 1.96(1.96) | 0.56 | 2.11(2.14) | 0.42 | 2.01(1.96) | <0.7 |
| 2128.983 | 26.96859 | 0.27 | 1.97(1.97) | 0.49 | 1.91(1.93) | 0.42 | 1.92(1.98) | 0.26 | 1.86(1.92) | <0.7 |
| 2132.909 | 25.82955 | 0.76 | 2.51(2.54) | 0.94 | 3.01(3.10) | 0.67 | 2.78(2.90) | 0.48 | 2.53(2.51) | <0.7 |
| 2135.958 | 25.80025 | 0.67 | 2.65(2.74) | 0.25 | 2.64(2.60) | 0.56 | 2.61(2.63) | 0.58 | 2.59(2.57) | 0.721796 |
| 2137.942 | 21.79294 | 0.9 | 2.97(3.01) | 0.91 | 2.91(2.91) | 0.89 | 2.87(2.94) | 0.76 | 2.77(2.77) | <0.7 |
| 2152.979 | 32.81957 | 0.5 | 2.06(2.06) | 0.25 | 2.06(2.05) | 0.37 | 2.03(2.11) | 0.44 | 2.25(2.34) | <0.7 |
| 2156.974 | 22.21732 | 0.83 | 2.75(2.81) | 0.86 | 2.84(2.93) | 0.85 | 2.82(2.91) | 0.74 | 2.59(2.57) | <0.7 |
| 2168.967 | 32.9057 | 0.55 | 2.65(2.71) | 0.66 | 2.58(2.63) | 0.86 | 2.68(2.72) | 0.76 | 2.66(2.65) | <0.7 |
| 2184.568 | 35.08425 | 0.55 | 2.58(2.65) | 0.88 | 2.69(2.74) | 0.63 | 2.53(2.57) | 0.34 | 2.61(2.59) | <0.7 |
| 2185.983 | 25.87921 | 0.9 | 3.20(3.31) | 0.89 | 3.08(3.16) | 0.92 | 3.09(3.21) | 0.68 | 2.96(3.06) | 0.731593 |
| 2187.949 | 39.78272 | 0.86 | 2.96(3.01) | 0.96 | 3.14(3.19) | 0.8 | 2.94(2.99) | 0.64 | 2.77(2.88) | <0.7 |
| 2188.999 | 26.88501 | 0.82 | 2.90(2.93) | 0.76 | 2.77(2.78) | 0.82 | 2.72(2.69) | 0.5 | 2.97(3.03) | <0.7 |
| 2191.99 | 22.39346 | 0.6 | 2.51(2.52) | 0.74 | 2.65(2.69) | 0.68 | 2.49(2.62) | 0.44 | 2.51(2.53) | <0.7 |
| 2194.971 | 20.16669 | 0.38 | 2.24(2.19) | 0.45 | 2.34(2.32) | 0.43 | 2.35(2.33) | 0.2 | 2.19(2.09) | <0.7 |
| 2198.999 | 22.31204 | 0.54 | 2.37(2.43) | 0.4 | 2.14(2.13) | 0.43 | 2.20(2.22) | 0.42 | 2.11(2.13) | <0.7 |
| 2208.891 | 31.70258 | 0.35 | 2.45(2.45) | 0.66 | 2.46(2.48) | 0.54 | 2.27(2.29) | 0.86 | 2.90(2.86) | <0.7 |
| 2211.975 | 33.23364 | 0.62 | 2.68(2.79) | 0.45 | 2.60(2.64) | 0.31 | 2.39(2.43) | 0.28 | 2.22(2.37) | <0.7 |
| 2216.03 | 33.83058 | 0.73 | 2.19(2.24) | 0.79 | 2.34(2.38) | 0.68 | 2.21(2.21) | 0.58 | 2.30(2.33) | 0.839981 |
| 2226.992 | 26.27752 | 0.67 | 4.00(4.05) | 0.8 | 4.15(4.14) | 0.79 | 4.02(4.07) | 0.52 | 4.02(4.17) | 0.723406 |
| 2233.045 | 20.51498 | 0.79 | 2.61(2.66) | 0.64 | 2.59(2.66) | 0.58 | 2.54(2.52) | 0.5 | 2.47(2.57) | <0.7 |
| 2235.045 | 34.16645 | 0.7 | 2.53(2.54) | 0.76 | 2.58(2.61) | 0.84 | 2.57(2.57) | 0.58 | 2.55(2.55) | 0.715329 |
| 2236.982 | 27.13894 | 0.78 | 2.69(2.77) | 0.84 | 2.59(2.59) | 0.8 | 2.42(2.40) | 0.48 | 2.15(2.38) | 0.750560 |
| 2249.042 | 20.532 | 0.74 | 2.64(2.66) | 0.69 | 2.60(2.69) | 0.62 | 2.51(2.61) | 0.48 | 2.36(2.47) | <0.7 |
| 2256.973 | 33.5541 | 0.5 | 2.60(2.58) | 0.43 | 2.69(2.69) | 0.64 | 2.56(2.63) | 0.52 | 2.57(2.62) | 0.749848 |
| 2257.869 | 35.92739 | 0.4 | 2.64(2.71) | 0.5 | 2.85(2.88) | 0.4 | 2.59(2.68) | 0.22 | 2.92(2.90) | <0.7 |
| 2258.185 | 22.08701 | 0.13 | 3.07(3.09) | 0.04 | 2.24(2.02) | 0.16 | 2.71(2.64) | 0.52 | 3.37(3.31) | 0.701593 |
| 2264.035 | 22.67238 | 0.88 | 2.68(2.74) | 0.8 | 2.56(2.61) | 0.8 | 2.50(2.53) | 0.76 | 2.36(2.40) | 0.778525 |
| 2265.974 | 33.73205 | 0.54 | 2.32(2.34) | 0.65 | 2.43(2.51) | 0.73 | 2.36(2.38) | 0.64 | 2.34(2.29) | <0.7 |
| 2266.021 | 22.1634 | 0.96 | 3.64(3.68) | 0.97 | 3.55(3.61) | 0.94 | 3.49(3.61) | 0.86 | 3.46(3.57) | 0.722271 |
| 2272.226 | 23.87656 | 0.71 | 2.40(2.45) | 0.5 | 2.12(2.15) | 0.59 | 2.22(2.23) | 0.5 | 2.13(2.10) | <0.7 |
| 2274.044 | 33.50617 | 0.23 | 2.10(2.18) | 0.4 | 2.22(2.24) | 0.37 | 2.03(2.07) | 0.34 | 1.89(1.95) | <0.7 |
| 2276.023 | 27.23118 | 0.56 | 3.22(3.52) | 0.67 | 3.56(3.64) | 0.57 | 3.36(3.54) | 0.62 | 3.24(3.46) | 0.706722 |
| 2277.008 | 27.22479 | 0.61 | 3.16(3.30) | 0.53 | 3.12(3.31) | 0.54 | 3.01(3.23) | 0.56 | 2.77(2.92) | <0.7 |
| 2280.944 | 36.21864 | 0.19 | 2.15(2.11) | 0.25 | 2.40(2.45) | 0.21 | 2.25(2.38) | 0.22 | 2.01(2.08) | <0.7 |
| 2282.016 | 22.23937 | 0.84 | 2.81(2.88) | 0.87 | 2.80(2.86) | 0.74 | 2.73(2.77) | 0.58 | 2.54(2.50) | 0.780767 |
| 2289.039 | 33.59018 | 0.29 | 2.02(2.09) | 0.53 | 2.31(2.36) | 0.48 | 2.08(2.13) | 0.32 | 1.97(2.08) | <0.7 |
| 2292.019 | 27.28272 | 0.98 | 3.41(3.45) | 1 | 3.58(3.61) | 0.95 | 3.40(3.46) | 0.92 | 3.36(3.47) | 0.735362 |
| 2302.198 | 26.13343 | 0.19 | 3.12(3.18) | 0.06 | 2.79(2.58) | 0.31 | 3.21(3.28) | 0.48 | 3.71(3.81) | <0.7 |
| 2308.017 | 27.33705 | 0.4 | 2.00(1.94) | 0.57 | 2.25(2.25) | 0.46 | 2.15(2.17) | 0.34 | 2.13(2.20) | 0.734816 |
| 2310.056 | 41.31365 | 0.41 | 2.20(2.31) | 0.77 | 2.33(2.37) | 0.37 | 2.13(2.12) | 0.42 | 2.10(2.20) | 0.712111 |
| 2318.21 | 26.30227 | 0.12 | 2.75(2.65) | 0.06 | 2.78(2.63) | 0.3 | 2.68(2.83) | 0.54 | 3.31(3.38) | 0.721003 |
| 2321.165 | 22.05962 | 0.62 | 2.41(2.41) | 0.49 | 2.44(2.47) | 0.47 | 2.36(2.27) | 0.24 | 2.38(2.26) | <0.7 |
| 2323.043 | 22.36064 | 0.83 | 2.63(2.71) | 0.74 | 2.50(2.54) | 0.71 | 2.58(2.67) | 0.68 | 2.46(2.62) | <0.7 |
| 2339 | 34.00721 | 0.81 | 2.59(2.63) | 0.92 | 2.84(2.89) | 0.89 | 2.76(2.80) | 0.74 | 2.58(2.70) | 0.735180 |
| 2349.043 | 27.36547 | 0.14 | 2.30(2.44) | 0.27 | 2.33(2.40) | 0.24 | 2.07(2.13) | 0.1 | 2.36(2.37) | <0.7 |
| 2355.085 | 22.74837 | 1 | 3.18(3.13) | 0.98 | 2.86(2.83) | 0.96 | 3.14(3.14) | 0.9 | 2.88(3.02) | <0.7 |
| 2356.659 | 35.533 | 0.81 | 2.55(2.64) | 0.97 | 2.83(2.86) | 0.8 | 2.54(2.60) | 0.64 | 2.58(2.70) | 0.730541 |
| 2361.108 | 20.79425 | 0.39 | 2.01(2.01) | 0.41 | 2.22(2.34) | 0.43 | 2.25(2.29) | 0.4 | 2.15(2.13) | <0.7 |
| 2371.084 | 22.7883 | 0.69 | 2.54(2.55) | 0.71 | 2.24(2.24) | 0.58 | 2.28(2.28) | 0.34 | 2.18(2.28) | 0.757463 |
| 2377.101 | 20.79967 | 0.95 | 2.93(3.01) | 0.91 | 3.12(3.22) | 0.88 | 2.97(3.05) | 0.7 | 2.84(2.85) | <0.7 |
| 2385.054 | 33.94877 | 0.76 | 2.59(2.58) | 0.84 | 2.72(2.77) | 0.79 | 2.63(2.72) | 0.68 | 2.55(2.65) | 0.728225 |
| 2389.241 | 22.39921 | 0.37 | 3.17(2.94) | 0.24 | 2.78(2.60) | 0.53 | 3.33(3.51) | 0.58 | 4.13(4.37) | <0.7 |
| 2394.08 | 23.63506 | 0.61 | 2.54(2.52) | 0.27 | 2.36(2.38) | 0.31 | 2.47(2.52) | 0.2 | 2.26(2.38) | 0.713591 |
| 2405.222 | 22.47371 | 0.29 | 2.73(2.52) | 0.15 | 2.42(2.17) | 0.44 | 2.92(2.80) | 0.62 | 3.72(3.78) | 0.724897 |
| 2407.092 | 27.67152 | 0.93 | 2.61(2.65) | 0.93 | 2.66(2.67) | 0.79 | 2.50(2.56) | 0.7 | 2.54(2.58) | <0.7 |
| 2414.154 | 19.57439 | 0.56 | 2.62(2.60) | 0.29 | 2.48(2.51) | 0.38 | 2.67(2.76) | 0.32 | 2.85(2.78) | <0.7 |
| 2414.626 | 35.61674 | 0.74 | 2.55(2.57) | 0.76 | 2.76(2.82) | 0.67 | 2.54(2.51) | 0.62 | 2.49(2.67) | <0.7 |
| 2420.998 | 34.86081 | 0.16 | 1.80(1.86) | 0.28 | 1.89(1.90) | 0.37 | 1.87(1.93) | 0.3 | 2.00(1.94) | 0.737104 |
| 2421.228 | 22.5266 | 0.1 | 2.74(2.63) | 0.04 | 2.66(2.69) | 0.2 | 2.63(2.65) | 0.56 | 3.20(3.20) | 0.754985 |
| 2423.092 | 27.66597 | 0.9 | 2.39(2.43) | 0.88 | 2.48(2.50) | 0.78 | 2.33(2.37) | 0.68 | 2.19(2.29) | <0.7 |
| 2423.324 | 21.07748 | 0.18 | 2.76(2.74) | 0.16 | 2.62(2.64) | 0.13 | 2.36(2.31) | 0.52 | 3.23(2.96) | 0.707434 |
| 2427.184 | 19.58138 | 0.08 | 2.39(2.58) | 0.09 | 3.14(2.71) | 0.14 | 2.56(2.43) | 0.56 | 3.11(3.03) | 0.742773 |
| 2430.098 | 28.32809 | 0.87 | 2.48(2.54) | 0.88 | 2.55(2.60) | 0.63 | 2.38(2.46) | 0.76 | 2.42(2.46) | <0.7 |
| 2446.092 | 28.37261 | 0.59 | 2.14(2.13) | 0.58 | 2.28(2.28) | 0.38 | 2.05(2.08) | 0.54 | 2.10(2.19) | <0.7 |
| 2471.155 | 34.77354 | 0.81 | 2.43(2.41) | 0.91 | 2.53(2.55) | 0.8 | 2.39(2.41) | 0.64 | 2.32(2.30) | <0.7 |
| 2485.125 | 34.4072 | 0.36 | 1.79(1.76) | 0.66 | 2.05(2.05) | 0.38 | 2.06(2.01) | 0.34 | 2.02(1.99) | <0.7 |
| 2490.231 | 24.68039 | 0.67 | 2.19(2.19) | 0.65 | 2.36(2.41) | 0.43 | 2.09(2.17) | 0.28 | 2.01(2.04) | <0.7 |
| 2501.119 | 34.38645 | 0.47 | 1.87(1.85) | 0.72 | 2.21(2.23) | 0.47 | 1.93(2.03) | 0.34 | 1.97(1.92) | 0.703610 |
| 2507.126 | 22.80579 | 0.58 | 2.41(2.43) | 0.55 | 2.17(2.21) | 0.56 | 2.11(2.13) | 0.36 | 2.34(2.35) | <0.7 |
| 2518.312 | 22.7888 | 0.22 | 3.02(3.26) | 0.09 | 2.75(2.69) | 0.36 | 3.12(3.21) | 0.54 | 3.74(3.88) | 0.707021 |
| 2525.195 | 27.73635 | 0.73 | 2.69(2.72) | 0.57 | 2.63(2.69) | 0.6 | 2.51(2.53) | 0.42 | 2.27(2.40) | 0.703540 |
| 2529.135 | 28.24742 | 0.58 | 2.39(2.47) | 0.31 | 2.11(2.07) | 0.37 | 2.22(2.19) | 0.32 | 2.35(2.39) | <0.7 |
| 2534.298 | 22.86796 | 0.11 | 2.44(2.40) | 0.04 | 2.62(2.34) | 0.2 | 2.91(3.03) | 0.62 | 3.11(3.35) | 0.775516 |
| 2540.264 | 19.67573 | 0.1 | 2.85(2.63) | 0.08 | 2.98(2.68) | 0.18 | 2.75(2.76) | 0.6 | 3.44(3.39) | 0.763953 |
| 2544.128 | 28.25992 | 0.34 | 2.12(2.21) | 0.55 | 2.25(2.25) | 0.28 | 2.15(2.28) | 0.2 | 2.40(2.32) | <0.7 |
| 2545.12 | 28.20161 | 0.67 | 2.66(2.66) | 0.47 | 2.40(2.36) | 0.38 | 2.50(2.44) | 0.48 | 2.40(2.44) | <0.7 |
| 2547.986 | 21.4417 | 0.65 | 2.51(2.53) | 0.65 | 2.53(2.60) | 0.52 | 2.46(2.44) | 0.24 | 2.26(2.33) | 0.746608 |
| 2548.286 | 35.15708 | 0.57 | 2.35(2.38) | 0.47 | 2.54(2.51) | 0.36 | 2.47(2.59) | 0.28 | 2.19(2.28) | <0.7 |
| 2559.18 | 19.40742 | 0.76 | 3.15(3.23) | 0.57 | 3.02(3.07) | 0.67 | 3.06(3.17) | 0.54 | 3.08(2.99) | <0.7 |
| 2563.147 | 21.21006 | 0.9 | 2.88(2.91) | 0.8 | 2.70(2.85) | 0.77 | 2.92(2.98) | 0.76 | 2.66(2.76) | <0.7 |
| 2564.15 | 22.97496 | 1 | 3.75(3.79) | 0.96 | 3.47(3.54) | 0.98 | 3.62(3.71) | 0.86 | 3.41(3.59) | 0.752743 |
| 2565.143 | 23.74013 | 0.56 | 2.51(2.53) | 0.17 | 2.12(2.06) | 0.34 | 2.57(2.66) | 0.52 | 2.51(2.57) | <0.7 |
| 2570.19 | 42.56018 | 0.85 | 3.73(3.82) | 0.91 | 3.86(3.91) | 0.74 | 3.54(3.64) | 0.62 | 3.68(3.71) | <0.7 |
| 2574.009 | 32.80843 | 0.21 | 1.93(1.92) | 0.53 | 1.91(1.95) | 0.28 | 1.84(1.95) | 0.4 | 1.92(1.92) | <0.7 |
| 2576.124 | 34.25753 | 0.31 | 1.79(1.81) | 0.35 | 2.05(2.11) | 0.23 | 1.76(1.74) | 0.26 | 1.74(1.80) | <0.7 |
| 2577.246 | 24.66592 | 0.96 | 2.61(2.67) | 0.87 | 2.55(2.64) | 0.79 | 2.40(2.52) | 0.74 | 2.40(2.38) | 0.737086 |
| 2580.141 | 22.9806 | 0.96 | 3.09(3.13) | 0.95 | 2.84(2.87) | 0.83 | 2.94(2.98) | 0.64 | 2.69(2.72) | 0.726339 |
| 2582.171 | 23.66997 | 0.53 | 2.34(2.39) | 0.42 | 2.16(2.20) | 0.3 | 2.13(2.18) | 0.34 | 2.06(2.06) | <0.7 |
| 2583.149 | 23.68335 | 0.36 | 2.43(2.44) | 0.4 | 2.25(2.26) | 0.32 | 2.27(2.32) | 0.3 | 2.36(2.38) | <0.7 |
| 2583.199 | 28.31444 | 0.88 | 2.98(3.03) | 0.8 | 2.64(2.73) | 0.72 | 2.66(2.78) | 0.72 | 2.65(2.67) | 0.716613 |
| 2584.234 | 35.18279 | 0.92 | 2.62(2.65) | 0.93 | 2.78(2.87) | 0.81 | 2.61(2.65) | 0.74 | 2.43(2.40) | 0.708466 |
| 2587.195 | 21.09996 | 0.36 | 2.21(2.19) | 0.48 | 2.38(2.42) | 0.38 | 2.44(2.43) | 0.18 | 2.38(2.46) | 0.714511 |
| 2589.056 | 22.56216 | 0.3 | 1.83(1.80) | 0.38 | 1.95(1.96) | 0.34 | 1.95(1.99) | 0.24 | 1.95(1.99) | <0.7 |
| 2596.233 | 34.89552 | 0.13 | 1.53(1.54) | 0.37 | 1.85(1.83) | 0.24 | 1.66(1.78) | 0.34 | 1.78(1.90) | 0.731331 |
| 2599.19 | 28.27509 | 0.91 | 2.77(2.79) | 0.84 | 2.51(2.54) | 0.76 | 2.27(2.22) | 0.76 | 2.32(2.37) | 0.721973 |
| 2636.2 | 24.39431 | 0.58 | 2.33(2.36) | 0.35 | 2.29(2.30) | 0.38 | 2.18(2.20) | 0.26 | 2.17(2.26) | <0.7 |
| 2639.289 | 21.41891 | 0.6 | 2.39(2.36) | 0.42 | 2.22(2.29) | 0.5 | 2.31(2.33) | 0.26 | 2.19(2.38) | <0.7 |
| 2642.214 | 27.69602 | 0.92 | 2.66(2.69) | 0.84 | 2.49(2.55) | 0.77 | 2.52(2.56) | 0.66 | 2.48(2.50) | <0.7 |
| 2644.217 | 21.15259 | 0.49 | 2.28(2.27) | 0.33 | 2.46(2.44) | 0.52 | 2.48(2.56) | 0.2 | 2.47(2.58) | <0.7 |
| 2654.193 | 23.92493 | 0.95 | 2.63(2.62) | 0.89 | 2.37(2.40) | 0.8 | 2.40(2.40) | 0.72 | 2.46(2.60) | <0.7 |
| 2663.204 | 23.50693 | 0.92 | 2.89(2.91) | 0.82 | 2.58(2.64) | 0.83 | 2.83(2.83) | 0.8 | 2.57(2.61) | <0.7 |
| 2679.197 | 23.52889 | 0.94 | 3.48(3.48) | 0.93 | 3.17(3.26) | 0.92 | 3.32(3.39) | 0.9 | 3.14(3.20) | 0.734395 |
| 2682.143 | 22.49183 | 0.93 | 2.85(2.91) | 0.93 | 2.92(2.97) | 0.81 | 2.75(2.78) | 0.64 | 2.72(2.86) | 0.733215 |
| 2686.336 | 29.34243 | 0.59 | 2.35(2.37) | 0.46 | 2.22(2.19) | 0.38 | 2.14(2.20) | 0.24 | 2.14(2.18) | <0.7 |
| 2687.219 | 28.98551 | 0.41 | 1.91(1.94) | 0.48 | 1.95(2.03) | 0.32 | 1.84(1.83) | 0.34 | 1.91(2.02) | <0.7 |
| 2695.198 | 23.52252 | 0.99 | 3.63(3.62) | 0.97 | 3.26(3.34) | 0.99 | 3.46(3.51) | 0.98 | 3.24(3.31) | 0.773776 |
| 2702.213 | 38.0799 | 0.7 | 2.47(2.55) | 0.66 | 2.43(2.50) | 0.65 | 2.25(2.35) | 0.36 | 2.23(2.29) | 0.705310 |
| 2710.323 | 35.07814 | 0.5 | 2.05(2.06) | 0.42 | 2.34(2.38) | 0.39 | 2.16(2.18) | 0.2 | 2.14(2.16) | <0.7 |
| 2713.234 | 29.22274 | 0.35 | 1.96(2.00) | 0.41 | 2.16(2.15) | 0.22 | 2.02(1.95) | 0.34 | 2.01(1.97) | <0.7 |
| 2726.283 | 42.93932 | 0.87 | 3.46(3.54) | 0.92 | 3.61(3.68) | 0.65 | 3.44(3.57) | 0.66 | 3.25(3.30) | <0.7 |
| 2733.781 | 34.1596 | 0.42 | 2.16(2.15) | 0.76 | 2.39(2.37) | 0.5 | 2.18(2.23) | 0.46 | 2.11(2.27) | 0.714863 |
| 2742.251 | 42.14319 | 0.71 | 2.79(2.90) | 0.88 | 2.81(2.89) | 0.6 | 2.51(2.65) | 0.52 | 2.60(2.69) | <0.7 |
| 2742.25 | 28.98028 | 0.95 | 2.97(3.06) | 0.96 | 2.94(3.05) | 0.86 | 2.92(2.98) | 0.84 | 2.81(2.86) | <0.7 |
| 2748.788 | 36.38231 | 0.46 | 1.78(1.81) | 0.75 | 2.27(2.32) | 0.49 | 1.77(1.82) | 0.46 | 2.01(2.01) | 0.748624 |
| 2752.413 | 19.8887 | 0.15 | 2.57(2.47) | 0.13 | 3.40(3.69) | 0.38 | 3.08(2.91) | 0.62 | 3.95(3.57) | 0.729735 |
| 2754.272 | 29.67942 | 0.81 | 2.52(2.63) | 0.88 | 2.58(2.63) | 0.54 | 2.38(2.46) | 0.48 | 2.26(2.35) | <0.7 |
| 2756.268 | 35.24034 | 0.51 | 2.13(2.11) | 0.72 | 2.33(2.36) | 0.57 | 2.20(2.28) | 0.44 | 2.03(1.93) | 0.705722 |
| 2761.315 | 21.49226 | 0.77 | 2.99(3.08) | 0.43 | 2.61(2.57) | 0.64 | 2.93(2.91) | 0.52 | 2.91(3.01) | <0.7 |
| 2767.323 | 21.6729 | 0.66 | 2.42(2.48) | 0.41 | 2.31(2.32) | 0.55 | 2.32(2.35) | 0.3 | 2.20(2.37) | 0.720059 |
| 2770.246 | 29.3928 | 0.51 | 2.05(2.08) | 0.22 | 1.82(1.88) | 0.31 | 1.98(1.91) | 0.34 | 1.81(1.79) | <0.7 |
| 2802.82 | 36.33852 | 0.43 | 2.04(2.07) | 0.6 | 2.53(2.56) | 0.39 | 2.11(2.09) | 0.46 | 2.33(2.29) | <0.7 |
| 2808.343 | 24.39073 | 0.66 | 2.88(2.97) | 0.23 | 2.65(2.74) | 0.31 | 2.75(2.90) | 0.26 | 2.43(2.46) | <0.7 |
| 2809.235 | 24.40412 | 0.39 | 2.71(2.85) | 0.76 | 2.80(2.81) | 0.57 | 2.71(2.76) | 0.66 | 2.60(2.72) | <0.7 |
| 2818.803 | 36.27764 | 0.34 | 2.14(2.13) | 0.6 | 2.54(2.61) | 0.21 | 2.33(2.25) | 0.26 | 2.42(2.23) | <0.7 |
| 2841.256 | 24.53535 | 0.82 | 3.16(3.25) | 0.63 | 2.90(2.93) | 0.72 | 2.81(2.84) | 0.72 | 2.63(2.73) | 0.721635 |
| 2887.347 | 35.71764 | 0.83 | 2.44(2.53) | 0.76 | 2.25(2.27) | 0.63 | 2.38(2.41) | 0.52 | 2.15(2.17) | 0.761239 |
| 2903.361 | 35.70914 | 0.54 | 2.08(2.16) | 0.4 | 1.84(1.80) | 0.41 | 1.84(1.79) | 0.5 | 1.69(1.65) | <0.7 |
| 2907.352 | 35.95513 | 0.81 | 2.51(2.56) | 0.77 | 2.27(2.30) | 0.69 | 2.28(2.35) | 0.52 | 2.29(2.29) | 0.739381 |
| 2914.379 | 24.33308 | 0.73 | 2.48(2.57) | 0.61 | 2.31(2.35) | 0.56 | 2.38(2.47) | 0.62 | 2.35(2.40) | <0.7 |
| 2923.432 | 36.91513 | 0.61 | 2.22(2.28) | 0.52 | 2.23(2.34) | 0.46 | 2.17(2.20) | 0.38 | 2.26(2.24) | <0.7 |
| 2926.3 | 22.21827 | 0.62 | 2.73(2.76) | 0.58 | 2.36(2.38) | 0.43 | 2.65(2.70) | 0.36 | 2.51(2.65) | <0.7 |
| 2939.149 | 33.76711 | 0.29 | 1.93(1.89) | 0.65 | 2.27(2.41) | 0.35 | 2.12(2.19) | 0.3 | 2.02(2.03) | <0.7 |
| 2942.299 | 22.23281 | 0.96 | 3.70(3.73) | 0.98 | 3.50(3.54) | 0.95 | 3.54(3.64) | 0.96 | 3.33(3.45) | 0.801121 |
| 2973.452 | 24.3704 | 0.79 | 2.77(2.84) | 0.57 | 2.49(2.60) | 0.66 | 2.63(2.64) | 0.48 | 2.56(2.58) | 0.703599 |
| 2977.373 | 29.11797 | 0.69 | 2.25(2.27) | 0.51 | 2.23(2.28) | 0.52 | 2.22(2.27) | 0.32 | 1.99(2.13) | 0.716047 |
| 2987.348 | 38.54569 | 0.31 | 2.47(2.37) | 0.6 | 2.36(2.39) | 0.43 | 2.30(2.28) | 0.54 | 2.43(2.39) | <0.7 |
| 2989.451 | 24.42708 | 0.7 | 2.62(2.74) | 0.32 | 2.39(2.40) | 0.5 | 2.36(2.36) | 0.36 | 2.35(2.44) | <0.7 |
| 2999.285 | 22.24535 | 0.82 | 3.42(3.45) | 0.94 | 3.10(3.15) | 0.74 | 3.20(3.35) | 0.7 | 3.08(3.10) | 0.754218 |
| 3002.238 | 23.80085 | 0.31 | 1.91(1.94) | 0.46 | 1.91(1.85) | 0.38 | 1.96(2.08) | 0.3 | 2.12(2.26) | <0.7 |
| 3007.407 | 20.95133 | 0.54 | 2.63(2.66) | 0.46 | 2.41(2.42) | 0.41 | 2.53(2.53) | 0.26 | 2.15(2.18) | <0.7 |
| 3013.292 | 22.2946 | 0.89 | 3.27(3.39) | 0.83 | 3.45(3.55) | 0.7 | 3.39(3.62) | 0.64 | 3.13(3.43) | <0.7 |
| 3021.351 | 23.41584 | 0.84 | 3.00(3.06) | 0.91 | 3.09(3.10) | 0.71 | 2.89(2.99) | 0.62 | 2.58(2.71) | 0.731622 |
| 3023.407 | 20.94963 | 0.6 | 2.39(2.39) | 0.43 | 2.29(2.29) | 0.46 | 2.28(2.34) | 0.22 | 2.36(2.33) | <0.7 |
| 3031.427 | 36.03612 | 0.66 | 2.17(2.15) | 0.65 | 2.22(2.25) | 0.58 | 2.17(2.21) | 0.44 | 2.11(2.14) | <0.7 |
| 3041.375 | 29.98314 | 0.95 | 3.52(3.54) | 0.98 | 3.34(3.43) | 0.85 | 3.17(3.32) | 0.92 | 3.14(3.22) | 0.707412 |
| 3058.378 | 24.82364 | 0.77 | 2.61(2.69) | 0.73 | 2.27(2.32) | 0.64 | 2.45(2.61) | 0.58 | 2.38(2.49) | 0.736932 |
| 3064.322 | 20.57415 | 0.42 | 2.47(2.44) | 0.52 | 2.49(2.57) | 0.53 | 2.64(2.67) | 0.34 | 2.50(2.42) | <0.7 |
| 3081.416 | 29.83057 | 0.21 | 2.14(2.14) | 0.14 | 2.19(2.09) | 0.14 | 2.09(2.10) | 0.38 | 2.28(2.27) | <0.7 |
| 3091.436 | 28.39639 | 0.73 | 2.46(2.51) | 0.89 | 2.71(2.78) | 0.69 | 2.34(2.43) | 0.58 | 2.41(2.47) | <0.7 |
| 3092.464 | 31.24934 | 0.86 | 2.50(2.53) | 0.88 | 2.55(2.60) | 0.65 | 2.49(2.55) | 0.72 | 2.44(2.52) | <0.7 |
| 3108.454 | 31.28399 | 0.82 | 2.44(2.45) | 0.89 | 2.52(2.59) | 0.72 | 2.40(2.45) | 0.62 | 2.36(2.42) | <0.7 |
| 3137.411 | 30.34616 | 0.81 | 2.74(2.76) | 0.74 | 2.53(2.58) | 0.55 | 2.48(2.57) | 0.68 | 2.44(2.53) | 0.718646 |
| 3139.487 | 29.48159 | 0.88 | 3.06(3.14) | 0.89 | 3.02(3.10) | 0.66 | 2.95(3.00) | 0.72 | 2.89(2.93) | <0.7 |
| 3145.459 | 38.89295 | 0.85 | 2.96(3.04) | 0.94 | 3.18(3.24) | 0.79 | 2.87(2.97) | 0.66 | 2.72(2.75) | <0.7 |
| 3149.46 | 31.24549 | 0.7 | 2.38(2.42) | 0.76 | 2.60(2.69) | 0.59 | 2.49(2.56) | 0.6 | 2.34(2.35) | 0.787247 |
| 3152.34 | 24.55108 | 0.86 | 3.00(3.03) | 0.71 | 2.65(2.73) | 0.63 | 2.82(2.90) | 0.74 | 2.78(2.83) | 0.750556 |
| 3157.128 | 34.69761 | 0.34 | 1.76(1.74) | 0.68 | 2.03(2.09) | 0.43 | 1.78(1.90) | 0.24 | 1.85(1.93) | 0.716292 |
| 3165.462 | 31.32057 | 0.58 | 2.20(2.26) | 0.73 | 2.43(2.51) | 0.46 | 2.32(2.35) | 0.46 | 2.20(2.27) | 0.753728 |
| 3166.271 | 22.05894 | 0.73 | 2.78(2.84) | 0.76 | 2.79(2.88) | 0.67 | 2.58(2.68) | 0.24 | 2.65(2.44) | <0.7 |
| 3168.357 | 24.70453 | 0.86 | 2.81(2.86) | 0.72 | 2.55(2.62) | 0.82 | 2.69(2.78) | 0.64 | 2.77(2.87) | <0.7 |
| 3193.382 | 22.64363 | 0.38 | 2.90(2.94) | 0.57 | 2.73(2.77) | 0.52 | 2.87(3.04) | 0.46 | 2.67(2.73) | 0.716663 |
| 3205.273 | 19.65755 | 0.79 | 2.69(2.78) | 0.72 | 2.86(2.92) | 0.63 | 2.70(2.76) | 0.62 | 2.49(2.46) | <0.7 |
| 3209.407 | 22.67502 | 0.97 | 3.73(3.79) | 0.97 | 3.68(3.71) | 0.9 | 3.56(3.63) | 0.78 | 3.24(3.36) | 0.819469 |
| 3256.527 | 33.03419 | 0.68 | 2.97(3.01) | 0.81 | 2.95(3.04) | 0.67 | 2.81(2.85) | 0.26 | 2.64(2.77) | 0.716578 |
| 3258.463 | 22.91476 | 0.91 | 3.23(3.27) | 0.87 | 3.03(3.17) | 0.84 | 3.13(3.18) | 0.82 | 2.88(2.90) | 0.720059 |
| 3264.556 | 25.75167 | 0.79 | 2.83(2.92) | 0.68 | 2.60(2.71) | 0.61 | 2.71(2.79) | 0.64 | 2.65(2.76) | <0.7 |
| 3264.528 | 30.65823 | 0.67 | 2.27(2.31) | 0.77 | 2.36(2.39) | 0.43 | 2.31(2.33) | 0.42 | 2.06(2.06) | <0.7 |
| 3266.417 | 22.74809 | 0.95 | 3.62(3.68) | 0.9 | 3.16(3.21) | 0.83 | 3.38(3.51) | 0.76 | 3.25(3.34) | 0.801062 |
| 3280.556 | 25.81551 | 0.88 | 2.60(2.64) | 0.75 | 2.43(2.48) | 0.66 | 2.53(2.58) | 0.54 | 2.60(2.59) | <0.7 |
| 3281.434 | 36.0914 | 0.97 | 3.35(3.41) | 0.95 | 3.11(3.20) | 0.91 | 3.19(3.22) | 0.88 | 2.98(3.10) | <0.7 |
| 3295.533 | 25.45276 | 0.59 | 2.32(2.32) | 0.31 | 2.01(1.99) | 0.43 | 2.24(2.21) | 0.3 | 2.15(2.27) | <0.7 |
| 3303.501 | 30.88648 | 0.81 | 2.70(2.72) | 0.72 | 2.52(2.53) | 0.64 | 2.45(2.56) | 0.7 | 2.21(2.28) | 0.702754 |
| 3314.431 | 20.14047 | 0.66 | 3.03(3.10) | 0.63 | 2.84(2.97) | 0.46 | 2.84(2.87) | 0.44 | 2.79(2.84) | <0.7 |
| 3318.546 | 30.99198 | 0.32 | 2.20(2.28) | 0.26 | 1.88(1.88) | 0.29 | 2.06(2.00) | 0.22 | 1.90(1.78) | 0.703722 |
| 3333.719 | 23.83213 | 0.52 | 2.68(2.72) | 0.35 | 2.49(2.53) | 0.45 | 2.55(2.61) | 0.38 | 2.55(2.64) | <0.7 |
| 3338.463 | 23.58844 | 0.73 | 2.98(3.02) | 0.81 | 2.86(2.89) | 0.58 | 2.86(2.92) | 0.58 | 2.77(2.80) | <0.7 |
| 3343.569 | 31.85325 | 0.33 | 2.35(2.41) | 0.39 | 2.32(2.36) | 0.41 | 2.30(2.35) | 0.28 | 2.20(2.25) | <0.7 |
| 3349.42 | 35.89379 | 0.61 | 2.37(2.40) | 0.57 | 2.23(2.31) | 0.52 | 2.24(2.25) | 0.46 | 1.98(1.90) | <0.7 |
| 3356.518 | 22.03026 | 0.12 | 2.99(2.98) | 0.09 | 3.25(3.03) | 0.15 | 2.65(2.80) | 0.58 | 3.30(3.38) | 0.728850 |
| 3359.578 | 31.89787 | 0.85 | 2.78(2.84) | 0.81 | 2.94(2.98) | 0.79 | 2.77(2.86) | 0.8 | 2.56(2.60) | 0.794521 |
| 3361.556 | 24.24354 | 0.51 | 2.09(2.07) | 0.51 | 2.17(2.15) | 0.35 | 1.99(1.98) | 0.14 | 1.83(1.91) | <0.7 |
| 3375.574 | 31.91691 | 0.91 | 2.73(2.83) | 0.92 | 2.92(2.97) | 0.81 | 2.77(2.84) | 0.72 | 2.67(2.72) | <0.7 |
| 3385.547 | 25.48872 | 0.88 | 3.59(3.63) | 0.69 | 3.22(3.37) | 0.82 | 3.36(3.48) | 0.54 | 3.25(3.38) | <0.7 |
| 3401.596 | 25.47106 | 0.89 | 3.20(3.25) | 0.7 | 2.87(3.01) | 0.88 | 3.06(3.08) | 0.66 | 2.74(2.80) | <0.7 |
| 3401.66 | 23.49281 | 0.72 | 2.86(2.93) | 0.8 | 2.72(2.76) | 0.54 | 2.58(2.71) | 0.38 | 2.48(2.49) | 0.732507 |
| 3416.602 | 36.84899 | 0.34 | 1.59(1.60) | 0.35 | 1.71(1.74) | 0.36 | 1.89(1.93) | 0.2 | 1.51(1.46) | <0.7 |
| 3421.555 | 25.99412 | 0.93 | 3.36(3.37) | 0.8 | 3.01(3.14) | 0.85 | 3.15(3.22) | 0.8 | 2.98(3.14) | 0.713630 |
| 3426.31 | 27.69928 | 0.49 | 2.05(2.10) | 0.69 | 2.24(2.32) | 0.5 | 2.12(2.13) | 0.4 | 1.96(2.08) | <0.7 |
| 3432.593 | 32.04683 | 0.94 | 3.01(3.05) | 0.95 | 2.96(3.00) | 0.91 | 2.94(3.03) | 0.86 | 2.79(2.81) | 0.701740 |
| 3478.434 | 41.74144 | 0.5 | 2.64(2.76) | 0.62 | 2.62(2.72) | 0.29 | 2.44(2.64) | 0.2 | 2.48(2.62) | <0.7 |
| 3510.6 | 40.24415 | 0.38 | 2.26(2.36) | 0.68 | 2.37(2.42) | 0.38 | 2.15(2.19) | 0.28 | 1.83(1.99) | <0.7 |
| 3530.645 | 26.12897 | 0.71 | 2.86(2.97) | 0.59 | 2.77(2.84) | 0.69 | 2.73(2.73) | 0.42 | 2.37(2.34) | 0.729499 |
| 3556.588 | 23.95474 | 0.81 | 2.94(3.05) | 0.76 | 2.86(2.90) | 0.6 | 2.74(2.86) | 0.7 | 2.66(2.69) | <0.7 |
| 3556.603 | 22.63602 | 0.19 | 3.22(3.17) | 0.2 | 2.72(2.69) | 0.27 | 3.04(3.19) | 0.64 | 3.63(3.72) | 0.723009 |
| 3559.709 | 24.92127 | 0.76 | 2.88(2.90) | 0.82 | 2.85(2.87) | 0.57 | 2.78(2.84) | 0.44 | 2.68(2.87) | 0.712271 |
| 3589.683 | 25.03383 | 0.82 | 2.71(2.75) | 0.87 | 2.77(2.84) | 0.63 | 2.73(2.83) | 0.52 | 2.52(2.48) | <0.7 |
| 3596.698 | 21.49245 | 0.56 | 2.56(2.59) | 0.31 | 2.39(2.41) | 0.41 | 2.37(2.39) | 0.28 | 2.25(2.22) | <0.7 |
| 3616.724 | 33.18799 | 0.75 | 2.59(2.62) | 0.79 | 2.53(2.54) | 0.71 | 2.44(2.47) | 0.5 | 2.48(2.62) | <0.7 |
| 3630.443 | 21.77706 | 0.73 | 2.73(2.76) | 0.6 | 2.56(2.65) | 0.47 | 2.46(2.58) | 0.34 | 2.65(2.58) | 0.718244 |
| 3635.661 | 31.79329 | 0.76 | 2.36(2.40) | 0.8 | 2.33(2.36) | 0.68 | 2.26(2.29) | 0.7 | 2.30(2.36) | <0.7 |
| 3657.665 | 40.71091 | 0.77 | 3.00(3.07) | 0.87 | 3.09(3.16) | 0.64 | 2.76(2.92) | 0.52 | 2.78(2.86) | <0.7 |
| 3669.666 | 24.16841 | 0.75 | 2.99(3.18) | 0.79 | 2.84(2.97) | 0.63 | 2.70(2.83) | 0.56 | 2.78(2.89) | <0.7 |
| 3685.833 | 22.19635 | 0.75 | 3.21(3.30) | 0.58 | 3.00(3.05) | 0.54 | 2.92(3.02) | 0.52 | 3.02(3.15) | 0.702537 |
| 3718.721 | 32.4816 | 0.88 | 2.67(2.67) | 0.86 | 2.54(2.57) | 0.8 | 2.62(2.67) | 0.8 | 2.46(2.53) | 0.778973 |
| 3719.734 | 22.49869 | 0.79 | 2.87(2.89) | 0.65 | 2.57(2.64) | 0.65 | 2.59(2.71) | 0.46 | 2.30(2.36) | 0.720219 |
| 3722.724 | 22.0386 | 0.81 | 3.52(3.61) | 0.61 | 3.07(3.07) | 0.59 | 3.08(3.16) | 0.5 | 2.92(3.02) | 0.731817 |
| 3734.721 | 32.49717 | 0.89 | 2.47(2.52) | 0.84 | 2.35(2.38) | 0.76 | 2.44(2.48) | 0.66 | 2.35(2.35) | <0.7 |
| 3738.721 | 24.76482 | 0.58 | 2.50(2.55) | 0.43 | 2.42(2.44) | 0.39 | 2.46(2.64) | 0.36 | 2.48(2.50) | <0.7 |
| 3745.661 | 26.68822 | 0.53 | 2.62(2.68) | 0.43 | 2.28(2.25) | 0.38 | 2.39(2.41) | 0.2 | 2.09(2.08) | <0.7 |
| 3759.851 | 19.41137 | 0.27 | 2.76(2.75) | 0.22 | 2.68(2.62) | 0.55 | 2.86(2.87) | 0.42 | 2.98(3.03) | <0.7 |
| 3774.719 | 22.93709 | 0.29 | 3.11(3.09) | 0.26 | 2.59(2.61) | 0.4 | 2.81(2.77) | 0.64 | 3.55(3.56) | 0.722655 |
| 3775.748 | 25.58515 | 0.88 | 2.98(3.03) | 0.91 | 3.04(3.05) | 0.78 | 2.82(2.91) | 0.54 | 2.62(2.64) | 0.754100 |
| 3802.714 | 32.43992 | 0.51 | 2.61(2.67) | 0.38 | 2.23(2.21) | 0.46 | 2.43(2.34) | 0.46 | 2.59(2.72) | <0.7 |
| 3816.753 | 21.96327 | 0.51 | 2.86(2.91) | 0.36 | 2.49(2.46) | 0.22 | 2.72(2.76) | 0.32 | 2.58(2.53) | <0.7 |
| 3839.813 | 19.70303 | 0.88 | 3.51(3.51) | 0.84 | 3.30(3.39) | 0.86 | 3.49(3.57) | 0.86 | 3.25(3.31) | 0.749848 |
| 3858.843 | 25.85393 | 0.77 | 2.99(3.01) | 0.79 | 2.96(3.02) | 0.63 | 2.83(2.83) | 0.52 | 2.87(3.06) | <0.7 |
| 3870.814 | 33.49116 | 0.24 | 1.87(1.88) | 0.46 | 2.05(2.05) | 0.28 | 1.81(1.86) | 0.2 | 1.79(1.81) | 0.725421 |
| 3886.83 | 33.53763 | 0.25 | 2.03(2.05) | 0.31 | 2.19(2.17) | 0.32 | 1.98(2.04) | 0.16 | 1.91(1.88) | <0.7 |
| 3891.752 | 24.52856 | 0.72 | 2.86(2.90) | 0.72 | 2.68(2.69) | 0.54 | 2.57(2.52) | 0.5 | 2.50(2.49) | <0.7 |
| 3927.821 | 33.59714 | 0.67 | 2.18(2.23) | 0.53 | 2.23(2.24) | 0.52 | 2.15(2.20) | 0.38 | 1.84(1.91) | <0.7 |
| 3932.864 | 25.93393 | 0.61 | 2.53(2.59) | 0.41 | 2.56(2.67) | 0.42 | 2.46(2.44) | 0.22 | 2.57(2.68) | <0.7 |
| 3943.83 | 33.62845 | 0.81 | 2.24(2.22) | 0.72 | 2.21(2.23) | 0.71 | 2.17(2.24) | 0.52 | 2.01(2.07) | 0.716431 |
| 3945.911 | 22.03043 | 0.56 | 2.99(3.05) | 0.37 | 2.69(2.82) | 0.33 | 2.79(2.84) | 0.24 | 2.56(2.68) | 0.721652 |
| 3968.597 | 21.09379 | 0.81 | 3.16(3.22) | 0.89 | 3.20(3.26) | 0.64 | 2.92(2.99) | 0.5 | 2.69(2.88) | 0.770973 |
| 3986.65 | 20.60164 | 0.97 | 3.66(3.67) | 0.91 | 3.57(3.64) | 0.81 | 3.32(3.48) | 0.7 | 3.17(3.19) | 0.728568 |
| 3996.658 | 20.92089 | 0.8 | 3.12(3.21) | 0.7 | 2.86(2.94) | 0.52 | 2.81(2.92) | 0.34 | 2.76(2.67) | 0.743766 |
| 4002.618 | 20.65664 | 0.46 | 2.55(2.46) | 0.47 | 2.68(2.72) | 0.35 | 2.69(2.75) | 0.44 | 2.90(3.03) | <0.7 |
| 4008.81 | 23.42187 | 0.3 | 3.19(3.25) | 0.26 | 2.59(2.53) | 0.42 | 3.01(2.96) | 0.62 | 3.65(3.63) | <0.7 |
| 4015.997 | 28.07419 | 0.59 | 2.50(2.61) | 0.49 | 2.43(2.48) | 0.44 | 2.46(2.51) | 0.38 | 2.18(2.26) | <0.7 |
| 4024.865 | 33.26167 | 0.59 | 2.42(2.46) | 0.52 | 2.37(2.35) | 0.48 | 2.34(2.39) | 0.5 | 2.28(2.27) | <0.7 |
| 4043.639 | 20.38493 | 0.82 | 3.11(3.08) | 0.77 | 3.05(3.09) | 0.73 | 2.88(3.03) | 0.52 | 2.70(2.75) | <0.7 |
| 4044.915 | 26.36705 | 0.76 | 3.35(3.41) | 0.85 | 3.26(3.29) | 0.69 | 3.08(3.14) | 0.52 | 2.98(2.90) | 0.719440 |
| 4083.799 | 31.25574 | 0.33 | 2.47(2.57) | 0.55 | 2.70(2.76) | 0.43 | 2.19(2.24) | 0.48 | 2.22(2.20) | <0.7 |
| 4097.87 | 24.61058 | 0.9 | 2.84(2.86) | 0.81 | 2.62(2.69) | 0.74 | 2.77(2.78) | 0.7 | 2.47(2.50) | 0.723274 |
| 4121.881 | 23.55676 | 0.19 | 3.18(3.29) | 0.1 | 2.66(2.55) | 0.35 | 3.06(3.13) | 0.52 | 3.50(3.58) | <0.7 |
| 4169.926 | 33.58317 | 0.93 | 3.01(3.05) | 0.92 | 3.00(3.05) | 0.9 | 2.88(2.98) | 0.84 | 2.83(2.81) | 0.702419 |
| 4217.975 | 26.05299 | 0.84 | 3.58(3.60) | 0.75 | 3.61(3.68) | 0.68 | 3.38(3.49) | 0.36 | 317(3.41) | 0.784425 |
| 4251.984 | 28.76518 | 0.75 | 2.85(2.88) | 0.62 | 2.70(2.81) | 0.63 | 2.70(2.82) | 0.6 | 2.24(2.36) | 0.731799 |
| 4305.937 | 28.82961 | 0.96 | 3.58(3.62) | 0.97 | 3.31(3.35) | 0.87 | 3.32(3.43) | 0.78 | 3.21(3.25) | <0.7 |
| 4321.941 | 25.19911 | 0.55 | 3.31(3.47) | 0.28 | 2.69(2.63) | 0.51 | 3.06(3.25) | 0.5 | 3.29(3.61) | <0.7 |
| 4352.86 | 20.16808 | 0.96 | 3.78(3.78) | 0.94 | 3.70(3.75) | 0.83 | 3.52(3.59) | 0.7 | 3.28(3.24) | 0.715144 |
| 4404.842 | 20.66586 | 0.71 | 2.99(2.95) | 0.78 | 2.94(3.01) | 0.56 | 2.79(2.84) | 0.32 | 2.63(2.44) | 0.767050 |
| 4409.888 | 20.00095 | 0.8 | 3.13(3.14) | 0.69 | 3.05(3.08) | 0.67 | 3.02(3.06) | 0.42 | 2.73(2.68) | <0.7 |
| 4436.083 | 26.31772 | 0.75 | 3.38(3.39) | 0.78 | 3.39(3.46) | 0.54 | 3.20(3.26) | 0.32 | 2.84(2.81) | 0.771622 |
| 4549.146 | 26.55078 | 0.5 | 3.27(3.32) | 0.37 | 2.93(2.99) | 0.27 | 3.10(3.25) | 0.22 | 2.67(2.90) | <0.7 |
| 4671.824 | 23.27783 | 0.67 | 2.56(2.63) | 0.73 | 2.62(2.65) | 0.53 | 2.50(2.59) | 0.38 | 2.44(2.55) | <0.7 |
| 4771.071 | 20.19867 | 0.73 | 3.14(3.18) | 0.71 | 2.95(2.97) | 0.54 | 2.90(2.97) | 0.3 | 2.68(2.76) | <0.7 |
| 4817.155 | 23.90309 | 0.54 | 3.16(3.32) | 0.11 | 2.64(2.64) | 0.36 | 3.06(3.31) | 0.46 | 3.02(3.27) | <0.7 |
| 4833.145 | 23.92431 | 0.59 | 3.11(3.22) | 0.17 | 2.54(2.56) | 0.43 | 3.04(3.17) | 0.38 | 3.14(3.21) | <0.7 |
| 4863.156 | 26.73903 | 0.74 | 2.59(2.68) | 0.65 | 2.31(2.34) | 0.54 | 2.54(2.66) | 0.38 | 2.44(2.50) | 0.707493 |
| 4960.418 | 20.61115 | 0.56 | 2.99(2.88) | 0.5 | 3.03(2.99) | 0.71 | 3.55(3.72) | 0.44 | 3.29(3.58) | 0.721512 |
| 5043.131 | 26.60105 | 0.56 | 2.78(2.84) | 0.27 | 2.28(2.29) | 0.38 | 2.44(2.65) | 0.24 | 2.49(2.56) | <0.7 |
| 5213.091 | 22.43408 | 0.53 | 2.29(2.28) | 0.63 | 2.33(2.33) | 0.47 | 2.30(2.35) | 0.3 | 2.11(2.18) | <0.7 |
| 5510.356 | 27.07841 | 0.66 | 2.64(2.70) | 0.71 | 2.59(2.61) | 0.63 | 2.48(2.50) | 0.4 | 2.27(2.30) | <0.7 |
| 5574.253 | 23.20092 | 0.76 | 2.74(2.75) | 0.84 | 2.79(2.87) | 0.77 | 2.94(2.98) | 0.38 | 2.66(2.64) | <0.7 |
| 6236.907 | 21.066 | 0.73 | 3.05(3.07) | 0.57 | 2.87(2.91) | 0.67 | 3.07(3.07) | 0.6 | 2.98(2.95) | 0.714541 |
| 6541.758 | 20.67838 | 0.5 | 2.81(2.74) | 0.31 | 2.51(2.52) | 0.22 | 2.70(2.71) | 0.3 | 2.62(2.61) | <0.7 |
| 8289.341 | 19.47861 | 0.75 | 3.10(3.10) | 0.64 | 3.01(3.06) | 0.47 | 2.97(3.09) | 0.58 | 2.92(2.95) | <0.7 |
| 8837.408 | 21.0634 | 0.72 | 3.41(3.50) | 0.71 | 3.16(3.31) | 0.63 | 3.40(3.42) | 0.64 | 3.10(3.22) | 0.702994 |
| 8853.766 | 21.09699 | 0.29 | 3.09(3.15) | 0.46 | 2.93(3.01) | 0.49 | 3.09(3.07) | 0.46 | 2.96(3.03) | <0.7 |
| 8917.251 | 22.54506 | 0.61 | 2.66(2.66) | 0.51 | 2.51(2.54) | 0.53 | 2.66(2.60) | 0.4 | 2.41(2.41) | <0.7 |
| 9866.536 | 20.86863 | 0.7 | 3.23(3.32) | 0.74 | 3.31(3.34) | 0.49 | 2.99(3.05) | 0.52 | 2.74(2.80) | 0.707316 |
| 10341.97 | 22.98239 | 0.51 | 3.40(3.60) | 0.53 | 3.07(3.18) | 0.52 | 3.01(3.13) | 0.32 | 2.99(3.21) | <0.7 |

RCC: Renal Cell Carcinoma.
Control: Normal control.
Bca: Bladder Carcinoma.
CKD: chronic kidney disease;
AUC. Area under the Curve

Preferably, at least five, at least six, at least eight, at least ten, at least 20 or at least 50 polypeptide markers or all markers as defined in Table 1 are used.

Preferably, markers are used whose molecular weight is 840 daltons or more. As an upper limit, markers are preferably suitable whose molecular weight is <10,000 daltons, more preferably <5000 daltons and even more preferably <4000 daltons. Due to the large number of polypeptides present in the urine, it is important that the markers are determined sufficiently exactly.

Preferably, the urine sample is a midstream urine sample.

The measurement of the amplitudes and/or presence or absence can be effected by a variety of methods. Suitable methods include capillary electrophoresis, HPLC, gas-phase ion spectrometry and/or mass spectrometry.

In a preferred embodiment, a capillary electrophoresis is performed before the molecular masses of the polypeptide markers are measured.

Mass spectrometry is particularly suitable for measuring the amplitude or presence or absence of the polypeptide marker or markers.

According to the invention, the process preferably has a sensitivity of at least 60% and a specificity of at least 60%. Preferably, the sensitivity is at least 70% or at least 80%, and the specificity is at least 70% or at least 80%.

In one embodiment of the invention, the sample is first separated into at least three, preferably at least 10 subsamples. This is followed by the analysis of at least three, preferably at least 10 subsamples for determining the presence or absence or amplitude of at least one polypeptide marker in the sample, wherein said polypeptide marker is selected from the markers of Table 1, which are characterized by their molecular masses and migration times (CE times).

The CE times stated in the Tables relate to a glass capillary of 90 cm in length and with an inner diameter (ID) of 50 μm at an applied voltage of 25 kV, and 20% acetonitrile, 0.25% formic acid in water is used as the mobile solvent. Details can be found in the experimental part.

### BRIEF DESCRIPTION OF THE DRAWINGS

FIG. 1 is a graphical representation of the data in table 1.

FIG. 2A shows ROC curves for the training set.

FIG. 2B shows ROC curves for the test set.

### DETAILED DESCRIPTION OF THE INVENTION

Specificity is defined as the number of actually negative samples divided by the sum of the numbers of the actually negative and false positive samples. A specificity of 100% means that a test recognizes all healthy persons as being healthy, i.e., no healthy subject is identified as being ill. This says nothing about how reliably the test recognizes sick patients.

Sensitivity is defined as the number of actually positive samples divided by the sum of the numbers of the actually positive and false negative samples. A sensitivity of 100% means that the test recognizes all sick persons. This says nothing about how reliably the test recognizes healthy patients.

By the markers according to the invention, it is possible to achieve a specificity of at least 60%, preferably at least 70%, more preferably at least 80%, even more preferably at least 90% and most preferably at least 95% for each of the stated diseases for which a diagnosis is desired.

By the markers according to the invention, it is possible to achieve a sensitivity of at least 60%, preferably at least 70%, more preferably at least 80%, even more preferably at least 90% and most preferably at least 95% for each of the stated diseases for which a diagnosis is desired.

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, 30% methanol, 0.5% formic acid in water is used, for example.

It is known that the CE migration times may vary. Nevertheless, the order in which the polypeptide markers are eluted is typically the same under the stated conditions for each CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 to 4 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.01% or ±0.005%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, in the course of the purification of the samples.

Proceeding from the parameters that determine the polypeptide markers (molecular weight and migration time), it is possible to identify the sequence of the corresponding polypeptides by methods known in the prior art.

The polypeptides according to the invention are used to diagnose renal cell carcinomas.

“Diagnosis” means the process of knowledge gaining by assigning symptoms or phenomena to a disease or injury. In the present case, the presence or absence of particular polypeptide markers is also used for differential diagnosis. The presence or absence of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present invention, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of a renal cell carcinoma. Thus, there are polypeptide markers which are typically present in patients with renal cell carcinomas, but do not or less frequently occur in subjects with no renal cell carcinoma. Further, there are polypeptide markers which are present in subjects with a renal cell carcinoma, but do not or less frequently occur in subjects with no renal cell carcinoma.

In addition or also alternatively to the frequency markers (determination of presence or absence), amplitude markers may also be used for diagnosis. Amplitude markers are used in such a way that the presence or absence is not critical, but the height of the signal (the amplitude) is decisive if the signal is present in both groups. In the Tables, the mean amplitudes of the corresponding signals (characterized by mass and migration time) averaged over all samples measured are stated. To achieve comparability between differently concentrated samples or different measuring methods, two normalization methods are possible. In the first approach, all peptide signals of a sample are normalized to a total amplitude of 1 million counts. Therefore, the respective mean amplitudes of the individual markers are stated as parts per million (ppm).

In addition, it is possible to define further amplitude markers by an alternative normalization method: In this case, all peptide signals of one sample are scaled with a common normalization factor, as set forth, for example, in Theodorescu et al. Electrophoresis, 26: 2797-808 (2005). Thus, a linear regression is formed between the peptide amplitudes of the individual samples and the reference values of all known polypeptides. The slope of the regression line just corresponds to the relative concentration and is used as a normalization factor for this sample.

All the groups employed consist of at least 20 individual patient or control samples in order to obtain a reliable mean amplitude. The decision for a diagnosis is made as a function of how high the amplitude of the respective polypeptide markers in the patient sample is in comparison with the mean amplitudes in the control groups or the “ill” group. If the value is in the vicinity of the mean amplitude of the “ill” group, the existence of a renal cell carcinoma is to be considered, and if it rather corresponds to the mean amplitudes of the control group, the non-existence of a renal cell carcinoma is to be considered. The distance from the mean amplitude can be interpreted as a probability of the sample's belonging to a certain group.

Alternatively, the distance between the measured value and the mean amplitude may be considered a probability of the sample's belonging to a certain group.

A frequency marker is a variant of an amplitude marker in which the amplitude is low in some samples. It is possible to convert such frequency markers to amplitude markers by including the corresponding samples in which the marker is not found into the calculation of the amplitude with a very small amplitude, on the order of the detection limit.

The subject from which the sample in which the presence or absence of one or more polypeptide markers is determined is derived may be any subject which is capable of suffering from renal cell carcinomas. Preferably, the subject is a mammal, and most preferably, it is a human.

In a preferred embodiment of the invention, not just three polypeptide markers, but a larger combination of markers are used to enable differential diagnosis. By comparing a plurality of polypeptide markers, a bias in the overall result due to a few individual deviations from the typical presence probability in the individual can be reduced or avoided.

The sample in which the presence or absence of the peptide marker or markers according to the invention is measured may be any sample which is obtained from the body of the subject. The sample is a sample which has a polypeptide composition suitable for providing information about the state of the subject. For example, it may be blood, urine, a synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a urine sample.

Urine samples can be taken as preferred in the prior art. Preferably, a midstream urine sample is used in the context of the present invention. For example, the urine sample may be taken by means of a catheter or also by means of a urination apparatus as described in WO 01/74275.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being measured is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) technique is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence or absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of suitable solvents include acetonitrile, methanol and the like. The solvents can be diluted with water or an acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electro-osmotic flow upon application of a current, while cations are accelerated towards the cathode, and anions are delayed. The advantage of capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are 30-100 cm. In addition, the capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection.

In the method according to the invention, it is advantageous to use several polypeptide markers for the diagnosis.

The use of at least 5, 6, 8 or 10 markers is preferred.

In one embodiment, from 20 to 50 markers are used.

In order to determine the probability of the existence of a disease when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication. Another possibility is the linear combination of individual signals as described, for example, in Rossing et al., J Am Soc Nephrol. (2008) 19(7): 1283-90.

### Example

### 1. Sample Preparation

For detecting the polypeptide markers for the diagnosis, urine was employed. Urine was collected from healthy donors (control group), from patients suffering from a chronic kidney disease or a bladder carcinoma (“diseases control”) as well as from patients suffering from a renal cell carcinoma.

For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μl of filtration buffer (2 M urea, 10 mM ammonia, 0.02% SDS). This 1.4 ml of sample volume was ultrafiltrated (20 kDa, Sartorius, Gottingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and desalted against 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a Beckman Coulter capillary electrophoresis system (P/ACE MDQ System; Beckman Coulter Inc., Fullerton, Calif., USA) and a Bruker ESI-TOF mass spectrometer (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 17 min. The total duration of a separation run was thus 65 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the following proteins or polypeptides which are characterized by the stated CE migration times under the chosen conditions were employed:

| Protein/polypeptide | Migration time |
|---|---|
| Aprotinin (SIGMA, Taufkirchen, DE, Cat. # A1153) | 19.3 min |
| Ribonuclease, SIGMA, Taufkirchen, DE, Cat. # R4875 | 19.55 min |
| Lysozyme, SIGMA, Taufkirchen, DE, Cat. # L7651 | 19.28 min |
| “REV”, Sequence: REVQSKIGYGRQIIS | 20.95 min |
| “ELM”, Sequence: ELMTGELPYSHINNRDQIIFMVGR | 23.49 min |
| “KINCON”, Sequence: TGSLPYSHIGSRDQIIFMVGR | 22.62 min |
| “GIVLY” Sequence: GIVLYELMTGELPYSHIN | 32.2 min |

The proteins/polypeptides were employed at a concentration of 10 pmol/μl each in water. “REV”, “ELM, “KINCON” and “GIVLY” are synthetic peptides.

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the invention. For example, they may proceed as follows: At first, they select one of the polypeptides found in their measurement (peptide 1) and try to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account.

Again, if several markers can be found with a corresponding mass, the most probable assignment is that in which there is a substantially linear relationship between the shift for peptide 1 and that for peptide 2.

Depending on the complexity of the assignment problem, it suggests itself to the skilled person to optionally use further proteins from their sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If they plot their measurement in the form of m/z versus migration time by means of a simple diagram (e.g., with MS Excel), the line patterns described also become visible. Now, a simple assignment of the individual polypeptides is possible by counting the lines.

Other approaches of assignment are also possible. Basically, the skilled person could also use the peptides mentioned above as internal standards for assigning their CE measurements.

### Testing the Markers

Urine samples from patients with RCC were analyzed to test the markers. Further data were based on urine samples obtained from 289 patients with non-RCC diseases and 310 healthy controls. The non-RCC diseases included among others patients with prostate concer, nephrovasculitis and lupus nephritis.

The samples were collected in two hospitals in Virginia, USA, and in Great Britain. Controls were also obtained from these two hospitals and from Hamburg, Hanover and Aachen.

The samples were subjected to the above described analysis.

On the one hand, the data found were used to identify the markers. A subgroup was used to determine a test set.

In this way, 517 biomarkers could be identified. In a test set of 34 RCC cases and 29 controls, a sensitivity of 82.4% and a specificity of 84.2% were found.

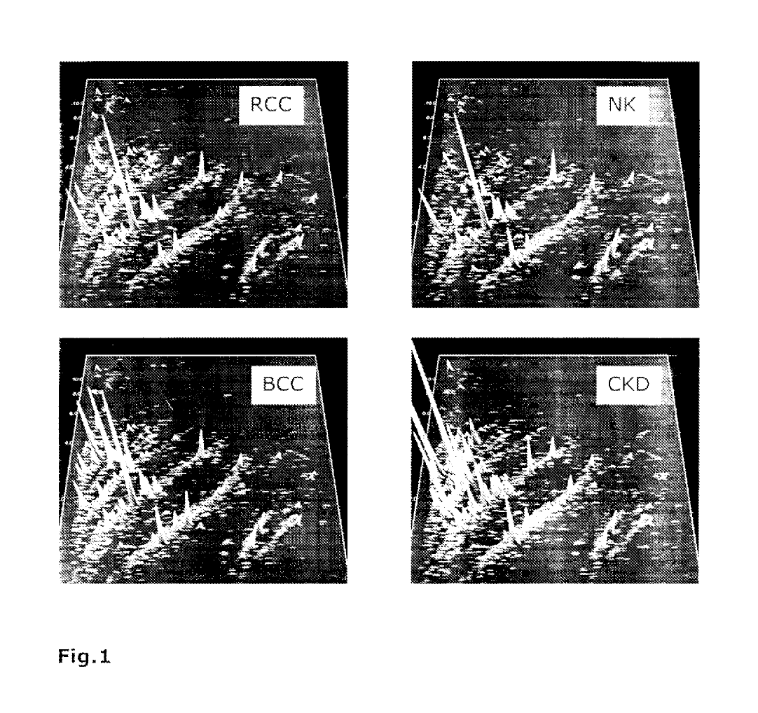

A graphical representation of the data is shown in FIG. 1.

NK=normal control
BCC=bladder cell carcinoma
CKD=chronic kidney disease.

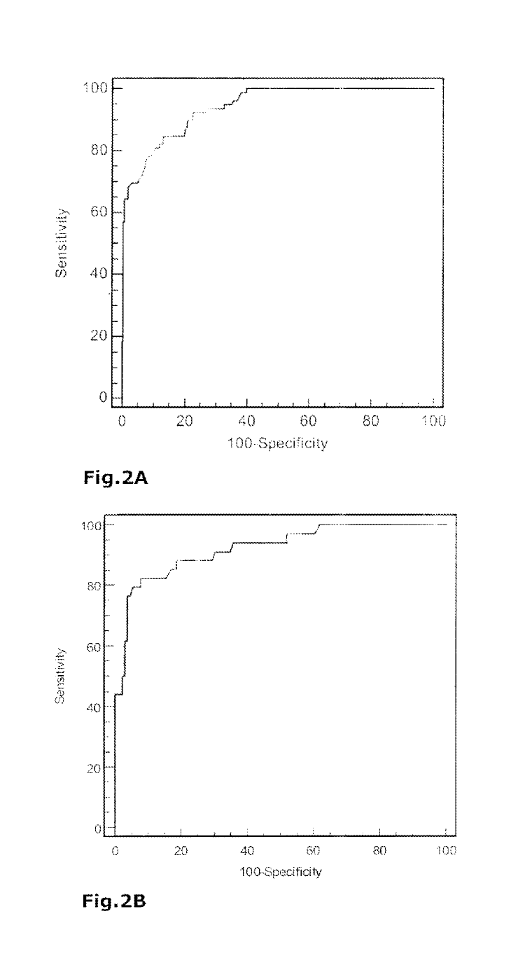

FIG. 2 shows the ROC curves for the training set and the test set.

The markers were in part sequenced. The sequence information obtained thereby is stated in Table 3.

TABLE 3

| | | | Start_ | Stop_ |
|---|---|---|---|---|
| Nr. | Sequence | Name | AA | AA |
| 1 | KGDTGPpGP | Collagen alpha-1 (III) chain | 629 | 637 |
| 2 | SpGEAGRpG | Collagen alpha-1 (I) chain | 522 | 530 |
| 13 | VLNLGPITR | Uromodulin | 598 | 606 |
| 19 | ApGDKGESGPS | Collagen alpha-1 (I) chain | 777 | 787 |
| 20 | PpGSAGAPGKDG | Collagen alpha-1 (I) chain | 1143 | 1154 |
| 22 | SpGPDGKTGPp | Collagen alpha-1 (I) chain | 546 | 556 |
| 23 | MGPRGPpGPpG | Collagen alpha-1 (I) chain | 217 | 227 |
| 25 | GpGSDGKpGPpG | Collagen alpha-1 (III) chain | 544 | 555 |
| 30 | ApGDRGEpGPp | Collagen alpha-1 (I) chain | 798 | 808 |
| 34 | GGpGSDGKpGPpG | Collagen alpha-1 (III) chain | 543 | 555 |
| 41 | GPpGpPGPPGPPS | Collagen alpha-1 (I) chain | 1181 | 1193 |
| 44 | PpGEAGKpGEQG | Collagen alpha-1 (I) chain | 651 | 662 |
| 45 | GPPGppGpPGPPS | Collagen alpha-1 (I) chain | 1181 | 1193 |
| 46 | MIEQNTKSPL | Alpha-1-antitrypsin | 398 | 407 |
| 53 | DDGEAGKpGRpG | Collagen alpha-1 (I) chain | 231 | 242 |
| 58 | GPpGEAGKpGEQG | Collagen alpha-1 (I) chain | 650 | 662 |
| 61 | ApGDRGEpGPPGP | Collagen alpha-1 (I) chain | 798 | 810 |
| 62 | DKGETGEQGDRG | Collagen alpha-1 (I) chain | 1095 | 1106 |
| 65 | SpGPDGKTGPpGPA | Collagen alpha-1 (I) chain | 546 | 559 |
| 71 | SpGSpGPDGKTGPp | Collagen alpha-1 (I) chain | 543 | 556 |
| 73 | TGPGGDKGDTGPpGP | Collagen alpha-1 (III) chain | 623 | 637 |
| 74 | SpGGpGSDGKpGPpG | Collagen alpha-1 (III) chain | 541 | 555 |
| 78 | DSGSSEEQGGSSRA | Polymeric-immunoglobulin | 626 | 639 |
| | | receptor | | |
| 85 | ApGEDGRpGPpGPQ | Collagen alpha-1 (II) chain | 511 | 524 |
| 86 | GSpGGpGSDGKpGPpG | Collagen alpha-1 (III) chain | 540 | 555 |
| 87 | DGPpGRDGQpGHKG | Collagen alpha-2 (I) chain | 933 | 946 |
| 96 | DEAGSEADHEGTHS | Fibrinogen alpha chain | 605 | 618 |
| 98 | ApGKNGERGGpGGpGP | Collagen alpha-1 (III) chain | 589 | 604 |
| 99 | YTKKVPQVSTPTL | Serum albumin | 435 | 447 |
| 101 | DQSRVLNLGPITR | Uromodulin | 594 | 606 |
| 103 | DGQPGAKGEpGDAGAK | Collagen alpha-1 (I) chain | 820 | 835 |
| 105 | DGQpGAKGEpGDAGAK | Collagen alpha-1 (I) chain | 820 | 835 |
| 108 | VGPpGpPGPPGPPGPPS | Collagen alpha-1 (I) chain | 1174 | 1190 |
| 110 | GSpGSpGPDGKTGPPGp | Collagen alpha-1 (I) chain | 542 | 558 |
| 113 | GDSDDDEPPPLPRL | Membrane associated | 54 | 67 |
| | | progesterone receptor | | |
| | | component 1 | | |
| 115 | VIDQSRVLNLGPIT | Uromodulin | 592 | 605 |
| 118 | DGQpGAKGEpGDAGAKG | Collagen alpha-1 (I) chain | 820 | 836 |
| 120 | GSEADHEGTHSTKRG | Fibrinogen alpha chain | 608 | 622 |
| 122 | YKRKANDESNEHS | Osteopontin | 246 | 258 |
| 124 | NDGApGKNGERGGpGGp | Collagen alpha-1 (III) chain | 586 | 602 |
| 126 | TGLSMDGGGSPKGDVDP | Na/K-ATPase gamma chain | 2 | 18 |
| 128 | ApGGKGDAGApGERGPpG | Collagen alpha-1 (III) chain | 670 | 687 |
| 130 | GPpGEAGKpGEQGVpGD | Collagen alpha-1 (I) chain | 650 | 666 |
| 132 | SGDSDDDEPPPLPRL | Membrane associated | 53 | 67 |
| | | progesterone receptor | | |
| | | component 1 | | |
| 133 | DGApGKNGERGGpGGpGP | Collagen alpha-1 (III) chain | 587 | 604 |
| 135 | MpGSpGGpGSDGKpGpPG | Collagen alpha-1 (III) chain | 538 | 555 |
| 136 | SpGNIGPAGKEGPVGLpG | Collagen alpha-2 (I) chain | 455 | 472 |
| 139 | GSpGSpGPDGKTGPpGPAG | Collagen alpha-1 (I) chain | 542 | 560 |
| 140 | AGSEADHEGTHSTKRG | Fibrinogen alpha chain | 607 | 622 |
| 144 | GLpGTGGPpGENGKPGEp | Collagen alpha-1 (III) chain | 642 | 659 |
| 145 | KpGEQGVpGDLGApGPSG | Collagen alpha-1 (I) chain | 657 | 674 |
| 147 | DEAGSEADHEGTHSTK | Fibrinogen alpha chain | 605 | 620 |
| 150 | GLpGTGGPpGENGKpGEp | Collagen alpha-1 (III) chain | 642 | 659 |
| 151 | PpGEAGKpGEQGVPGDLG | Collagen alpha-1 (I) chain | 651 | 668 |
| 163 | GPpGPpGKNGDDGEAGKpG | Collagen alpha-1 (I) chain | 221 | 239 |
| 168 | GPpGEAGKpGEQGVpGDLG | Collagen alpha-1 (I) chain | 650 | 668 |
| 174 | EEAPSLRPAPPPISGGGY | Fibronogen beta chain | 54 | 71 |
| 175 | GNDGAPGKNGERGGPGGPGP | Collagen alpha-1 (III) chain | 585 | 604 |
| 176 | GEKGPSGEAGTAGPpGTpGP | Collagen alpha-2 (I) chain | 844 | 863 |
| 182 | GLpGTGGPpGENGKPGEPGp | Collagen alpha-1 (III) chain | 642 | 661 |
| 184 | GpPGEGRAGEpGTAGpTGpP | Collagen alpha-2 (VIII) chain | 490 | 509 |
| 187 | GSVIDQSRVLNLGPITR | Uromodulin | 590 | 606 |
| 188 | DEAGSEADHEGTHSTKR | Fibrinogen alpha chain | 605 | 621 |
| 199 | NSGEpGApGSKGDTGAKGEp | Collagen alpha-1 (I) chain | 432 | 451 |
| 201 | EGSpGRDGSpGAKGDRGET | Collagen alpha-1 (I) chain | 1021 | 1039 |
| 202 | DAGPAGPKGEpGSpGENGApG | Collagen alpha-1 (I) chain | 279 | 299 |
| 204 | DDGEAGKPGRPGERGppGP | Collagen alpha-1 (I) chain | 231 | 249 |
| 217 | GDDGEAGKPGRpGERGPpGP | Collagen alpha-1 (I) chain | 230 | 249 |
| 219 | kGNDGApGKNGERGGpGGpGP | Collagen alpha-1 (III) chain | 584 | 604 |
| 221 | EAIPMSIPPEVKFNKPF | Alpha-1-antitrypsin | 378 | 394 |
| 223 | GDDGEAGkPGRpGERGPpGP | Collagen alpha-1 (I) chain | 230 | 249 |
| 233 | DGESGRPGRPGERGLPGPPG | Collagen alpha-1 (III) chain | 230 | 249 |
| 235 | AGpPGPPGppGTSGHpGSpGSpG | Collagen alpha-1 (III) chain | 176 | 198 |
| 237 | GEPGGkGERGApGEKGEGGpPG | Collagen alpha-1 (III) chain | 819 | 840 |
| 239 | PpGEAGKpGEQGVpGDLGAPGP | Collagen alpha-1 (I) chain | 648 | 669 |
| 241 | SGSVIDQSRVLNLGPITRK | Uromodulin | 589 | 607 |
| 247 | NGDDGEAGKpGRpGERGPpGP | Collagen alpha-1 (I) chain | 229 | 249 |
| 251 | GppGEAGKPGEQGVPGDLGAPGp | Collagen alpha-1 (I) chain | 647 | 669 |
| 255 | SNGNpGpPGPSGSPGKDGPpGpAG | Collagen alpha-1 (III) chain | 886 | 909 |
| 256 | DGKTGpPGPAGQDGRPGPpGppG | Collagen alpha-1 (I) chain | 550 | 572 |
| 259 | NGEpGGKGERGApGEKGEGGpPG | Collagen alpha-1 (III) chain | 818 | 840 |
| 261 | AEGSpGRDGSpGAKGDRGETGPA | Collagen alpha-1 (I) chain | 1020 | 1042 |
| 266 | ADGQPGAKGEPGDAGAKGDAGPpGP | Collagen alpha-1 (I) chain | 819 | 843 |
| 267 | NGDDGEAGkPGRpGERGPpGPQ | Collagen alpha-1 (I) chain | 229 | 250 |
| 268 | NDGPpGRDGQpGHKGERGYpG | Collagen alpha-2 (I) chain | 932 | 952 |
| 273 | GNSGEpGApGSKGDTGAKGEpGPVG | Collagen alpha-1 (I) chain | 431 | 455 |
| 274 | GKNGDDGEAGKPGRpGERGPpGP | Collagen alpha-1 (I) chain | 227 | 249 |
| 275 | GRTGDAGPVGPPGPpGppGpPGPPS | Collagen alpha-1 (I) chain | 1169 | 1193 |
| 277 | GKNGDDGEAGKpGRpGERGpPGP | Collagen alpha-1 (I) chain | 227 | 249 |
| 280 | IEQNTKSPLFMGKVVNPTQK | Alpha-1-antitrypsin; C-term. | 399 | 418 |
| 281 | KGDAGApGApGGKGDAGApGERGpPG | Collagen alpha-1 (III) chain | 662 | 687 |
| 283 | QNGEpGGKGERGAPGEKGEGGppG | Collagen alpha-1 (III) chain | 817 | 840 |
| 286 | ADGQPGAKGEpGDAGAKGDAGPPGpA | Collagen alpha-1 (I) chain | 819 | 844 |
| 291 | ADGQpGAKGEpGDAGAKGDAGPpGPA | Collagen alpha-1 (I) chain | 819 | 844 |
| 293 | ADGQpGAKGEpGDAGAKGDAGppGPA | Collagen alpha-1 (I) chain | 819 | 844 |
| 297 | GQNGEpGGKGERGApGEKGEGGPpG | Collagen alpha-1 (III) chain | 816 | 840 |
| 300 | KGNSGEpGApGSKGDTGAKGEpGPVG | Collagen alpha-1 (I) chain | 430 | 455 |
| 302 | GKNGDDGEAGKPGRpGERGPpGPQ | Collagen alpha-1 (I) chain | 227 | 250 |
| 304 | GKNGDDGEAGkPGRpGERGPpGPQ | Collagen alpha-1 (I) chain | 227 | 250 |
| 306 | MIEQNTKSPLFMGKVVNPTQK | Alpha-1-antitrypsin | 398 | |
| 313 | mIEQNTKSPLFmGKVVNPTQK | Alpha-1-antitrypsin | 398 | |
| 314 | LDGAKGDAGPAGPKGEPGSPGENGAPG | Collagen alpha-1 (I) chain | 273 | 299 |
| 316 | DAHKSEVAHRFKDLGEENFKA | Serum albumin; N-term. | 25 | 45 |
| 317 | ADGQPGAKGEpGDAGAKGDAGPpGPAGP | Collagen alpha-1 (I) chain | 819 | 846 |
| 318 | ADGQpGAKGEpGDAGAKGDAGpPGPAGP | Collagen alpha-1 (I) chain | 819 | 846 |
| 319 | TGPIGPpGPAGApGDKGESGPSGPAGPTG | Collagen alpha-1 (I) chain | 766 | 794 |
| 324 | LmIEQNTKSPLFMGKVVNPTQK | Alpha-1-antitrypsin; C-term. | 397 | 418 |
| 326 | GPPGADGQpGAKGEpGDAGAKGDAGpPGP | Collagen alpha-1 (I) chain | 815 | 843 |
| 328 | DAHKSEVAHRFKDLGEENFKAL | Serum albumin; N-term. | 25 | 46 |
| 330 | GPpGADGQpGAKGEpGDAGAKGDAGpPGP | Collagen alpha-1 (I) chain | 815 | 843 |
| 333 | DEAGSEADHEGTHSTKRGHAKSRP | Fibrinogen alpha chain | 605 | 628 |
| 335 | GApGQNGEpGGKGERGApGEKGEGGPpG | Collagen alpha-1 (III) chain | 813 | 840 |
| 340 | DDILASPPRLPEPQPYPGAPHHSS | Collagen alpha-1 (XVIII) chain | 1296 | 1319 |
| 342 | PEAEAEAEAGAGGEAAAEEGAAGRKARG | Zinc finger protein 653 | 8 | 35 |
| 344 | AGPpGApGApGAPGPVGPAGKSGDRGETGP | Collagen alpha-1 (I) chain | 1042 | 1071 |
| 349 | AGPpGApGApGApGPVGPAGKSGDRGETGP | Collagen alpha-1 (I) chain | 1042 | 1071 |
| 352 | QGpPGPSGEEGKRGPNGEAGSAGPPGppG | Collagen alpha-2 (I) chain | 369 | 397 |
| 353 | GEPLDARGHGRPGGSGASEEALSPRGAG | similar to Cyclin G-associated | 1246 | 1273 |
| | | kinase | | |
| 354 | ERGEAGIpGVpGAKGEDGKDGSpGEpGA | Collagen alpha-1 (III) chain | 448 | 475 |
| 356 | NRGERGSEGSPGHpGQpGppGpPGAPGP | Collagen alpha-1 (III) chain | 1168 | 1195 |
| 360 | NRGERGSEGSpGHpGQpGppGPPGAPGp | Collagen alpha-1 (III) chain | 1168 | 1195 |
| 367 | KNGETGPQGPpGPTGPGGDKGDTGPpGPQG | Collagen alpha-1 (III) chain | 610 | 639 |
| 369 | DAHKSEVAHRFKDLGEENFKALVL | Serum albumin; N-term. | 25 | 48 |
| 372 | ERGSPGpAGPKGSpGEAGRpGEAGLpGAKG | Collagen alpha-1 (I) chain | 510 | 539 |
| 373 | KEGGKGPRGETGPAGRpGEVGpPGPpGPAG | Collagen alpha-1 (I) chain | 903 | 932 |
| 374 | GPpGADGQpGAKGEpGDAGAKGDAGpPGP | Collagen alpha-1 (I) chain | 815 | 843 |
| 377 | ERGEAGIpGVpGAKGEDGKDGSPGEpGANG | Collagen alpha-1 (III) chain | 448 | 477 |
| 385 | ESGREGAPGAEGSpGRDGSpGAKGDRGETGP | Collagen alpha-1 (I) chain | 1011 | 1041 |
| 387 | ESGREGApGAEGSpGRDGSpGAKGDRGETGP | Collagen alpha-1 (I) chain | 1011 | 1041 |
| 392 | GESGREGApGAEGSpGRDGSpGAKGDRGETGP | Collagen alpha-1 (I) chain | 1010 | 1041 |
| 395 | ESGREGApGAEGSpGRDGSpGAKGDRGETGPA | Collagen alpha-1 (I) chain | 1011 | 1042 |
| 396 | DGVSGGEGKGGSDGGGSHRKEGEEADAPGVIPG | CD99 antigen | 97 | 129 |
| 405 | ADGQpGAKGEpGDAGAKGDAGpPGPAGPAGPPGp | Collagen alpha-1 (I) chain | 819 | 854 |
| | IG | | | |
| 409 | GADGQPGAKGEpGDAGAKGDAGPpGPAGpAGPP | Collagen alpha-1 (I) chain | 818 | 854 |
| | GPIG | | | |
| 414 | GEpGRDGVpGGpGMRGmpGSpGGpGSDGKpGPp | Collagen alpha-1 (III) chain | 522 | 555 |
| | G | | | |
| 415 | PpGESGREGAPGAEGSpGRDGSpGAKGDRGETG | Collagen alpha-1 (I) chain | 1008 | 1041 |
| | P | | | |
| 417 | PpGESGREGApGAEGSpGRDGSpGAKGDRGETGP | Collagen alpha-1 (I) chain | 1008 | 1041 |
| 419 | ENGKPGEpGpKGDAGApGApGGKGDAGApGERGp | Collagen alpha-1 (III) chain | 652 | 687 |
| | PG | | | |
| 421 | RTGEVGAVGPpGFAGEkGPSGEAGTAGPpGTpGP | Collagen alpha-2 (I) chain | 830 | 865 |
| | QG | | | |
| 422 | GPpGESGREGApGAEGSpGRDGSpGAKGDRGET | Collagen alpha-1 (I) chain | 1007 | 1041 |
| | GP | | | |
| 430 | EEKAVADTRDQADGSRASVDSGSSEEQGGSSRA | Polymeric-immunoglobulin | 607 | 639 |
| | | receptor | | |
| 431 | PpGADGQPGAKGEpGDAGAKGDAGpPGPAGPAG | Collagen alpha-1 (I) chain | 816 | 854 |
| | PPGPIG | | | |
| 433 | EDPQGDAAQKTDTSHHDQDHPTFNKITPNL | Alpha-1-antitrypsin; N-term. | 25 | 54 |
| 447 | FAEEKAVADTRDQADGSRASVDSGSSEEQGGSSR | Polymeric-immunoglobulin | 606 | 639 |
| | A | receptor | | |
| 448 | EDPQGDAAQKTDTSHHDQDHPTFNKITPNLAE | Alpha-1-antitrypsin; N-term. | 25 | 56 |
| 457 | GRPEAQPPPLSSEHKEPVAGDAVPGPKDGSAPEV | Neurosecretory protein VGF | 26 | 62 |
| | RGA | | | |
| 462 | EEKAVADTRDQADGSRASVDSGSSEEQGGSSRAL | Polymeric-immunoglobulin | 607 | 643 |
| | VST | receptor | | |
| 467 | ANGAPGNDGAKGDAGAPGAPGSQGAPGLQGMPG | Collagen alpha-1 (I) chain | 699 | 741 |
| | ERGAAGLPGP | | | |
| 490 | ERGEQGPAGSpGFQGLpGpAGppGEAGKpGEQGV | Collagen alpha-1 (I) chain | 630 | 674 |
| | PGDLGAPGPSG | | | |
| 491 | EEKAVADTRDQADGSRASVDSGSSEEQGGSSRAL | Polymeric-immunoglobulin | 607 | 648 |
| | VSTLVPLG | receptor | | |
| 492 | N/A | | | |
| 493 | ARGNDGARGSDGQpGpPGPpGTAGFpGSpGAKGE | Collagen alpha-1 (III) chain | 319 | 366 |
| | VGpAGSpGSNGApG | | | |
| 494 | LQGLPGTGGPPGENGKPGEPGPKGDAGAPGAPG | Collagen alpha-1 (III) chain | 640 | 687 |
| | GKGDAGAPGERGPPG | | | |

What is claimed is:

1. A process for the diagnosis of a renal cell carcinoma, comprising: measuring the presence or absence based on occurrence frequency measurements or concentration amplitude altered in a disease state of at least three polypeptide markers in a urine sample, wherein said polypeptide markers are selected from the markers as identified in Table 1; and comparing the presence or absence or amplitudes with corresponding values of a healthy population of patients, and with corresponding values of a population of patients with renal cell carcinoma to obtain the diagnosis.

2. The process according to claim 1, wherein said diagnosis is a differential diagnosis for distinguishing between a renal cell carcinoma and one or more diseases selected from chronic kidney diseases and bladder cancer.

3. The process according to claim 1, wherein an evaluation of the determined presence or absence or amplitudes of the markers is effected by means of the reference values of Table 2.

4. The process according to claim 1, wherein at least five polypeptide markers as defined in claim 1 are used.

5. The process according to claim 1, wherein the markers 1, 2, 13, 19, 20, 22, 23, 25, 30, 34, 41, 44, 45, 46, 53, 58, 61, 62, 65, 71, 73, 74, 78, 85, 86, 87, 96, 98, 99, 101, 103, 105, 108, 110, 113, 115, 118, 120, 122, 124, 126, 128, 130, 132, 133, 135, 136, 139, 140, 144, 145, 147, 150, 151, 163, 168, 174, 175, 176, 182, 184, 187, 188, 199, 201, 202, 204, 217, 219, 221, 223, 233, 235, 237, 239, 241, 247, 251, 255, 256, 259, 261, 266, 267, 268, 273, 274, 275, 277, 280, 281, 283, 286, 291, 293, 297, 300, 302, 304, 306, 313, 314, 316, 317, 318, 319, 324, 326, 328, 330, 333, 335, 340, 342, 344, 349, 352, 353, 354, 356, 360, 367, 369, 372, 373, 374, 377, 385, 387, 392, 395, 396, 405, 409, 414, 415, 417, 419, 421, 422, 430, 431, 433, 447, 448, 457, 462, 467, 490, 491, 493, 494 are used.

6. The process according to claim 1, wherein all the markers are used.

7. The process according to claim 1, wherein said urine sample is a midstream urine sample.

8. The process according to claim 1 wherein capillary electrophoresis and mass spectrometry are used for measuring the presence or absence or amplitude of the polypeptide markers.

9. The process according to claim 8 wherein the capillary electrophoresis is performed before the molecular mass of the polypeptide markers is measured.

10. The process according to claim 1 where the measuring step comprises:
a) separating the urine sample into at least three subsamples;
b) analyzing at least three subsamples by measuring the presence or absence or amplitude of at least one polypeptide marker in the sample, wherein said polypeptide marker is selected from the markers of Table 1.

11. A device for the quantitative evaluation of the polypeptide markers found in a urine sample, wherein said device comprises a data base containing data sets corresponding to reference values of polypeptides, and information about the presence or absence or amplitude of the polypeptides in samples from healthy or ill subjects, wherein said data base at least contains information relating to the identity of the markers and the presence and absence or amplitude of three polypeptide markers from Table 1.

12. The process according to claim 1, wherein at least six polypeptide markers as defined in claim 1 are used.

13. The process according to claim 1, wherein at least eight polypeptide markers as defined in claim 1 are used.

14. The process according to claim 1, wherein at least ten polypeptide markers as defined in claim 1 are used.

15. The process according to claim 1, at least 20 polypeptide markers as defined in claim 1 are used.

16. The process according to claim 1, wherein at least 50 polypeptide markers as defined in claim 1 are used.
